SEQUENCE LISTING (24 sequences)

SEQ ID NO: 1 - protein, 15 aa, Artificial
  Antibody Light Chain CDR
RASKSVSTSGFSYLH

SEQ ID NO: 2 - protein, 7 aa, Artificial
  Antibody Light Chain CDR
LASNLES

SEQ ID NO: 3 - protein, 9 aa, Artificial
  Antibody Light Chain CDR
QHSWELPLT

SEQ ID NO: 4 - protein, 5 aa, Artificial
  Antibody Heavy Chain CDR
SYYLY

SEQ ID NO: 5 - protein, 17 aa, Artificial
  Antibody Heavy Chain CDR
GVNPSNGGTNFSEKFKS

SEQ ID NO: 6 - protein, 11 aa, Artificial
  Antibody Heavy Chain CDR
RDSNYDGGFDY

SEQ ID NO: 7 - protein, 15 aa, Artificial
  Antibody Light Chain CDR
RASKGVSTSGYSYLH

SEQ ID NO: 8 - protein, 7 aa, Artificial
  Antibody Light Chain CDR
LASYLES

SEQ ID NO: 9 - protein, 9 aa, Artificial
  Antibody Light Chain CDR
QHSRDLPLT

SEQ ID NO: 10 - protein, 5 aa, Artificial
  Antibody Heavy Chain CDR
NYYMY

SEQ ID NO: 11 - protein, 17 aa, Artificial
  Antibody Heavy Chain CDR
GINPSNGGTNFNEKFKN

SEQ ID NO: 12 - protein, 11 aa, Artificial
  Antibody Heavy Chain CDR
RDYRFDMGFDY

SEQ ID NO: 13 - protein, 120 aa, Artificial [sequence omitted]

SEQ ID NO: 14 - protein, 447 aa, Artificial
  Humanized Antibody Heavy Chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 111 aa, Artificial [sequence omitted]

SEQ ID NO: 16 - protein, 111 aa, Artificial
  Humanized Antibody Light Chain Variable Region
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 17 - protein, 111 aa, Artificial
  Humanized Antibody Light Chain Variable Region
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 18 - protein, 218 aa, Artificial
  Humanized Antibody Light Chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 218 aa, Artificial
  Humanized Antibody Light Chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 218 aa, Artificial
  Humanized Antibody Light Chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 447 aa, Artificial; identical to SEQ ID NO: 14
  Humanized Antibody Heavy Chain

SEQ ID NO: 22 - protein, 218 aa, Artificial; identical to SEQ ID NO: 18
  Humanized Antibody Light Chain

SEQ ID NO: 23 - protein, 440 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 214 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2016/0312297 A1
(43) Pub. Date: Oct. 27, 2016

(54) PD-L1 GENE SIGNATURE BIOMARKERS OF TUMOR RESPONSE TO PD-1 ANTAGONISTS

(71) Applicant: Merck Sharp & Dohme Corp., Rahway, NJ (US)

(72) Inventors: Mark Ayers, Pennington, NJ (US); Andrey Loboda, Canton, MA (US); Jared Lunceford, Washington, UT (US); Terrill K. McClanahan, Sunnyvale, CA (US); Erin Murphy, Redwood City, CA (US); Michael Nebozhyn, Yeadon, PA (US); Robert H. Pierce, San Francisco, CA (US)

(73) Assignee: Merck Sharp & Dohme Corp., Rahway, NJ (US)

(21) Appl. No.: 15/104,539

(22) PCT Filed: Dec. 15, 2014
(86) PCT No.: PCT/US14/70237

§ 371 Date: Jun. 15, 2016

Related U.S. Application Data
(60) Provisional application No. 61/917,277, filed on Dec. 17, 2013.

(51) Int. Cl. C12Q 1/68; C07K 16/28

(52) U.S. Cl. CPC C12Q 1/6886; C07K 16/2818; C12Q 2600/158; C12Q 2600/106; C07K 2317/24

(57) ABSTRACT

The present disclosure describes PD-L1 gene signature biomarkers that are useful for identifying cancer patients who are most likely to benefit from treatment with a PD-1 antagonist. The disclosure also provides methods and kits for testing tumor samples for the biomarkers, as well as methods for treating subjects with a PD-1 antagonist based on the test results.

### FIELD OF THE INVENTION

The present invention relates generally to the treatment of cancer. In particular, the invention relates to methods for identifying patients who are likely to respond to treatment with an antagonist of Programmed Death 1 (PD-1).

### BACKGROUND OF THE INVENTION

PD-1 is recognized as an important player in immune regulation and the maintenance of peripheral tolerance. PD-1 is moderately expressed on naive T, B and NKT cells and up-regulated by T/B cell receptor signaling on lymphocytes, monocytes and myeloid cells (1).

Two known ligands for PD-1, PD-L1 (B7-H1) and PD-L2 (B7-DC), are expressed in human cancers arising in various tissues. In large sample sets of e.g. ovarian, renal, colorectal, pancreatic, liver cancers and melanoma, it was shown that PD-L1 expression correlated with poor prognosis and reduced overall survival irrespective of subsequent treatment (2-13). Similarly, PD-1 expression on tumor infiltrating lymphocytes was found to mark dysfunctional T cells in breast cancer and melanoma (14-15) and to correlate with poor prognosis in renal cancer (16). Thus, it has been proposed that PD-L1 expressing tumor cells interact with PD-1 expressing T cells to attenuate T cell activation and evasion of immune surveillance, thereby contributing to an impaired immune response against the tumor.

Several monoclonal antibodies that inhibit the interaction between PD-1 and one or both of its ligands PD-L1 and PD-L2 are in clinical development for treating cancer. These include nivolumab and MK-3475, which are antibodies that bind to PD-1, and MPDL3280A, which binds to PD-L1. While clinical studies with these antibodies have produced durable anti-tumor responses in some cancer types, a significant number of patients failed to exhibit an anti-tumor response. Thus, a need exists for diagnostic tools to identify which cancer patients are most likely to achieve a clinical benefit to treatment with a PD-1 antagonist.

An active area in cancer research is the identification of gene expression patterns, commonly referred to as gene signatures or molecular signatures, which are characteristic of particular types or subtypes of cancer, and which may be associated with clinical outcomes.

### SUMMARY OF THE INVENTION

The present invention provides PD-L1 gene signature biomarkers that are predictive of tumor response to therapy with PD-1 antagonists. A biomarker of the invention is a composite intratumoral RNA expression score (a “gene signature score”) for a PD-L1 gene signature which comprises PD-L1 and a specific set of about five to ten additional genes that are co-expressed with PD-L1 in multiple tumor types. The gene signature score for a tumor sample of interest is calculated as the arithmetic mean of normalized RNA expression levels, in the tumor sample, for each of the genes in the gene signature.

Typically, the tumor sample is from a subject who is treatment naïve for anti-PD-1 therapy. To assess whether such a subject's tumor is likely to respond to a PD-1 antagonist, the calculated score for the tumor sample is compared to a reference score for the PD-L1 gene signature that has been pre-selected to divide at least the majority of responders to anti-PD-1 therapy from at least the majority of non-responders to anti-PD-1 therapy. If the PD-L1 gene signature score for the tumor sample is equal to or a greater than the reference PD-L1 gene signature score, the subject is more likely to have an anti-tumor response, or to achieve a better anti-tumor response, than if the tumor sample score is less than the reference score.

The co-expressed genes comprising a PD-L1 gene signature of the invention are selected from the genes shown in Table 1 below.

TABLE 1
PD-L1 Co-expressed Genes

| | Target |
|---|---|
| Gene | Transcript |
| PDL-1 | NM_014143 |
| PDL-2 | NM_025239 |
| LAG3 | NM_002286 |
| STAT1 | NM_007315 |
| CXCL10 | NM_001565 |
| CLEC10a | NM_182906 |

An exemplary PD-L1 gene signature of the invention comprises PD-L1, PD-L2, STAT1, LAG3, CXCL10, and CLEC10a. However, the inventors contemplate that other predictive PD-L1 gene signatures may be developed by one or both of: (a) substituting at least one of the co-expressed genes (PD-L2, STAT1, LAG3, CXCL10, and CLEC10a) by a different gene that is co-expressed with PD-L1 as listed in Table 1; and (b) adding one or more additional PD-L1 co-expressed genes from Table 1. The inventors contemplate that determining a subject's PD-L1 gene signature score will be useful in a variety of research and clinical applications.

Thus, in one aspect, the invention provides a method for testing a tumor for the presence or absence of a biomarker that predicts response to treatment with a PD-1 antagonist. The method comprises obtaining a sample from the tumor, measuring the RNA expression level in the tumor sample for each gene in a PD-L1 gene signature, and calculating a score for the PD-L1 gene signature from the measured RNA expression levels. In some embodiments, the method further comprises comparing the calculated score to a reference score for the PD-L1 gene signature, and classifying the tumor as biomarker positive or biomarker negative. If the calculated score is equal to or greater than the reference score, then the tumor is classified as biomarker positive, and if the calculated PD-L1 gene signature score is less than the reference PD-L1 gene signature score, then the tumor is classified as biomarker negative.

In another aspect, the invention provides a method for treating a subject having a tumor which comprises determining if the tumor is positive or negative for a PD-L1 gene signature biomarker and administering to the subject a PD-1 antagonist if the tumor is positive for the biomarker and administering to the subject a cancer treatment that does not include a PD-1 antagonist if the tumor is negative for the biomarker.

In yet another aspect, the invention provides a method for treating a subject having a tumor which comprises obtaining a sample from the tumor, measuring the expression level in the tumor sample for each gene in a PD-L1 gene signature, calculating a score for the PD-L1 gene signature from the measured expression levels, and administering to the subject a PD-1 antagonist if the calculated score is equal to or greater than a reference score for the PD-L1 gene signature or administering to the subject a cancer therapy that does not contain a PD-1 antagonist if the calculated score is less than the reference score. In some preferred embodiments, the reference score is pre-selected to divide the majority of responders to the PD-1 antagonist from the majority of non-responders to the PD-1 antagonist. In other preferred embodiments, the reference score is pre-selected to divide the majority of good responders to the PD-1 antagonist from the majority of poor responders to the PD-1 antagonist.

In a still further aspect, the invention provides a pharmaceutical composition comprising a PD-1 antagonist for use in a subject who has a tumor that tests positive for a PD-L1 gene signature biomarker.

Yet another aspect of the invention is a drug product which comprises a pharmaceutical composition and prescribing information. The pharmaceutical composition comprises a PD-1 antagonist and at least one pharmaceutically acceptable excipient. The prescribing information states that the pharmaceutical composition is indicated for use in a subject who has a tumor that tests positive for a PD-L1 gene signature biomarker.

In another aspect, the invention provides a kit useful for assaying a tumor sample to determine a PD-L1 gene signature score for the tumor sample. The kit comprises a first set of probes for detecting expression of each gene in the PD-L1 gene signature. The kit comprises, for each target transcript in the gene signature, at least one probe for the target transcript. In some preferred embodiments, the target transcripts are the transcripts listed in Table 1 for PD-L1, PD-L2, STAT1, LAG3, CXCL10, and CLEC10a. In other preferred embodiments, the kit may also comprise a second set of probes for detecting expression of a set of normalization genes. The normalization gene set consists of 10 to 1000 genes, e.g., this gene set may consist of at least any of 25, 50, 75, 100, 150, 200, 300, 400, 500, 600, 700, 800 or 900 genes. The kit may also comprise a plurality of control tumor samples which may be assayed for expression of the PD-L1 gene signature and normalization genes in the same manner as the test tumor sample.

In some preferred embodiments of any of the above aspects of the invention, the PD-L1 gene signature consists essentially of PD-L1, PD-L2, STAT1, LAG3, CXCL10, and CLEC10a. In some particularly preferred embodiments, the test and reference PD-L1 gene signature scores are determined by performing quantile normalization of raw RNA expression values for the genes in the gene signature relative to the distribution of raw RNA expression values for a set of at least 200, 250, 300, 350 or 400 normalization genes, followed by a subsequent log 10-transformation. In such embodiments, the reference gene signature score is between 1.87 and 2.12, between 1.96 and 2.12, or is about 2.12.

In all of the above aspects and embodiments of the invention, the PD-1 antagonist inhibits the binding of PD-L1 to PD-1, and preferably also inhibits the binding of PD-L2 to PD-1. In some preferred embodiments, the PD-1 antagonist is a monoclonal antibody, or an antigen binding fragment thereof, which specifically binds to PD-1 or to PD-L1 and blocks the binding of PD-L1 to PD-1. In particularly preferred embodiments, the PD-1 antagonist is an anti-PD-1 antibody which comprises a heavy chain and a light chain, wherein the heavy and light chains comprise the amino acid sequences shown in FIG. 6 (SEQ ID NO:21 and SEQ ID NO:22).

In some embodiments of any of the above aspects of the invention, the subject is a human and the cancer is a solid tumor and in some preferred embodiments, the solid tumor is bladder cancer, breast cancer, clear cell kidney cancer, head/neck squamous cell carcinoma, lung squamous cell carcinoma, malignant melanoma, non-small-cell lung cancer (NSCLC), ovarian cancer, pancreatic cancer, prostate cancer, renal cell cancer, small-cell lung cancer (SCLC) or triple negative breast cancer. In some particularly preferred embodiments, the human subject has ipilimumab-naïve advanced melanoma, while in other particularly preferred embodiments the human subject has ipilimumab-refractory advanced melanoma.

In other particularly preferred embodiments of any of the above aspects of the invention, the tumor is metastatic melanoma, the PD-1 antagonist is MK-3475, the PD-L1 gene signature consists essentially of PD-L1, PD-L2, STAT1, LAG3, CXCL10, and CLEC10a, and the reference score is 2.1.

In other particularly preferred embodiments of any of the above aspects of the invention, a responder achieves a partial response (PR) or complete response (CR) as measured by RECIST 1.1 criteria, and a non-responder does not achieve either a PR or CR.

### BRIEF DESCRIPTION OF THE DRAWINGS

FIG. 1 shows amino acid sequences of the light chain and heavy chain CDRs for an exemplary anti-PD-1 monoclonal antibody useful in the present invention (SEQ ID NOs:1-6).

FIG. 2 shows amino acid sequences of the light chain and heavy chain CDRs for another exemplary anti-PD-1 monoclonal antibody useful in the present invention (SEQ ID NOs:7-12).

FIG. 3 shows amino acid sequences of the heavy chain variable region and full length heavy chain for an exemplary anti-PD-1 monoclonal antibody useful in the present invention (SEQ ID NO:13 and SEQ ID NO:14).

FIG. 4 shows amino acid sequences of alternative light chain variable regions for an exemplary anti-PD-1 monoclonal antibody useful in the present invention (SEQ ID NOs:15-17).

FIG. 5 shows amino acid sequences of alternative light chains for an exemplary anti-PD-1 monoclonal antibody useful in the present invention (SEQ ID NOs:18-20).

FIG. 6 shows amino acid sequences of the heavy and light chains for MK-3475 (SEQ ID NOs. 21 and 22, respectively).

FIG. 7 shows amino acid sequences of the heavy and light chains for nivolumab (SEQ ID NOs. 23 and 24, respectively).

FIG. 8 shows a scatter plot of scores for a preferred PD-L1 gene signature of the invention in pre-treatment melanoma tumor samples from a cohort of 19 patients classified according to their Responder Status to MK-3475 treatment as described in the Examples below, and shows the P value determined for a one-sided t-test analysis of the association between PD-L1 gene signature score and responder status in this cohort.

FIG. 9 shows a scatter plot of scores for a preferred PD-L1 gene signature of the invention in pre-treatment melanoma tumor samples from a cohort of 19 patients classified according to length in days of progression-free survival (PFS), with the open circles identifying patients who had no progression at the time of response evaluation, and the P value showing the result of a cox-regression analysis of association between PD-L1 gene signature score and PFS in response to MK-3475.

FIG. 10 shows a bar graph of response rates in a cohort of 19 melanoma patients treated with MK-3475 and who were classified as having either a low score or a high score for a preferred PD-L1 gene signature of the invention based on a reference score (cut-off) of 2.1.

FIG. 11 shows a box plot graph of PFS (in months) in a cohort of 19 melanoma patients treated with MK-3475 and who were classified as having either a low score or a high score for a preferred PD-L1 gene signature of the invention based on a reference score (cut-off) of 2.1, with the horizontal line in the box indicating the median, the top and bottom edges of the box representing the 25th and 75th percentiles, the whiskers extending to the most extreme data points not considered outliers, and outliers plotted individually.

### DETAILED DESCRIPTION

### Abbreviations

Throughout the detailed description and examples of the invention the following abbreviations will be used:

CDR Complementarity determining region
CHO Chinese hamster ovary
CLEC10a C-type lectin domain family 10 member A

### CR Complete Response

CXCL10 Chemokine (C-X-C motif) ligand 10
DFS Disease free survival
FFPE formalin-fixed, paraffin-embedded
FR Framework region

### IgG Immunoglobulin G

IHC Immunohistochemistry or immunohistochemical
LAG3 Lymphocyte activation gene 3
OR Overall response

### NCBI National Center for Biotechnology Information

OS Overall survival

### PD Progressive Disease

### PD-1 Programmed Death 1

### PD-L1 Programmed Cell Death 1 Ligand 1

### PD-L2 Programmed Cell Death 1 Ligand 2

PFS Progression free survival (PFS)

### PR Partial Response

Q2W One dose every two weeks
Q3W One dose every three weeks

### RECIST Response Evaluation Criteria in Solid Tumors

### SD Stable Disease

STAT1 Signal transducer and activator of transcription 1
VH Immunoglobulin heavy chain variable region
VK Immunoglobulin kappa light chain variable region

### I. DEFINITIONS

So that the invention may be more readily understood, certain technical and scientific terms are specifically defined below. Unless specifically defined elsewhere in this document, all other technical and scientific terms used herein have the meaning commonly understood by one of ordinary skill in the art to which this invention belongs.

As used herein, including the appended claims, the singular forms of words such as “a,” “an,” and “the,” include their corresponding plural references unless the context clearly dictates otherwise.

“About” when used to modify a numerically defined parameter (e.g., the gene signature score for a gene signature discussed herein, or the dosage of a PD-1 antagonist, or the length of treatment time with a PD-1 antagonist) means that the parameter may vary by as much as 10% above or below the stated numerical value for that parameter. For example, a gene signature consisting of about 10 genes may have between 9 and 11 genes. Similarly, a reference gene signature score of about 2.12 includes scores of 1.91, 2.33 and any score between 1.91 and 2.33.

“Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, dog, cat, rabbit) and most preferably a human.

As used herein, the term “antibody” refers to any form of antibody that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, chimeric antibodies and camelized single domain antibodies. “Parental antibodies” are antibodies obtained by exposure of an immune system to an antigen prior to modification of the antibodies for an intended use, such as humanization of an antibody for use as a human therapeutic.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

The variable regions of each light/heavy chain pair form the antibody binding site. Thus, in general, an intact antibody has two binding sites. Except in bifunctional or bispecific antibodies, the two binding sites are, in general, the same.

Typically, the variable domains of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3 and FR4. The assignment of amino acids to each domain is, generally, in accordance with the definitions of Sequences of Proteins of Immunological Interest, Kabat, et al.; National Institutes of Health, Bethesda, Md.; 5^{th} ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

As used herein, the term “hypervariable region” refers to the amino acid residues of an antibody that are responsible for antigen-binding. The hypervariable region comprises amino acid residues from a “complementarity determining region” or “CDR” (i.e. CDRL1, CDRL2 and CDRL3 in the light chain variable domain and CDRH1, CDRH2 and CDRH3 in the heavy chain variable domain). See Kabat et al. (1991) Sequences of Proteins of Immunological Interest, 5th Ed. Public Health Service, National Institutes of Health, Bethesda, Md. (defining the CDR regions of an antibody by sequence); see also Chothia and Lesk (1987) J. Mol. Biol. 196: 901-917 (defining the CDR regions of an antibody by structure). As used herein, the term “framework” or “FR” residues refers to those variable domain residues other than the hypervariable region residues defined herein as CDR residues.

As used herein, unless otherwise indicated, “antibody fragment” or “antigen binding fragment” refers to antigen binding fragments of antibodies, i.e. antibody fragments that retain the ability to bind specifically to the antigen bound by the full-length antibody, e.g. fragments that retain one or more CDR regions. Examples of antibody binding fragments include, but are not limited to, Fab, Fab′, F(ab′)₂, and Fv fragments; diabodies; linear antibodies; single-chain antibody molecules, e.g., sc-Fv; nanobodies and multispecific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g. without producing undesired results such as false positives. Antibodies, or binding fragments thereof, useful in the present invention will bind to the target protein with an affinity that is at least two fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g. the amino acid sequence of a mature human PD-1 or human PD-L1 molecule, if it binds to polypeptides comprising that sequence but does not bind to proteins lacking that sequence.

“Chimeric antibody” refers to an antibody in which a portion of the heavy and/or light chain is identical with or homologous to corresponding sequences in an antibody derived from a particular species (e.g., human) or belonging to a particular antibody class or subclass, while the remainder of the chain(s) is identical with or homologous to corresponding sequences in an antibody derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences only. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” is added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Biotherapeutic agent” means a biological molecule, such as an antibody or fusion protein, that blocks ligand/receptor signaling in any biological pathway that supports tumor maintenance and/or growth or suppresses the anti-tumor immune response.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include squamous cell carcinoma, myeloma, small-cell lung cancer, non-small cell lung cancer, glioma, hodgkin's lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, breast cancer, colon carcinoma, and head and neck cancer. Particularly preferred cancers that may be treated in accordance with the present invention include those characterized by elevated expression of one or both of PD-L1 and PD-L2 in tested tissue samples.

“CDR” or “CDRs” as used herein means complementarity determining region(s) in a immunoglobulin variable region, defined using the Kabat numbering system, unless otherwise indicated.

“Chemotherapeutic agent” is a chemical compound useful in the treatment of cancer. Classes of chemotherapeutic agents include, but are not limited to: alkylating agents, antimetabolites, kinase inhibitors, spindle poison plant alkaloids, cytoxic/antitumor antibiotics, topoisomerase inhibitors, photosensitizers, anti-estrogens and selective estrogen receptor modulators (SERMs), anti-progesterones, estrogen receptor down-regulators (ERDs), estrogen receptor antagonists, leutinizing hormone-releasing hormone agonists, anti-androgens, aromatase inhibitors, EGFR inhibitors, VEGF inhibitors, anti-sense oligonucleotides that that inhibit expression of genes implicated in abnormal cell proliferation or tumor growth. Chemotherapeutic agents useful in the treatment methods of the present invention include cytostatic and/or cytotoxic agents.

“Clothia” as used herein means an antibody numbering system described in Al-Lazikani et al., JMB 273:927-948 (1997).

“Conservatively modified variants” or “conservative substitution” refers to substitutions of amino acids in a protein with other amino acids having similar characteristics (e.g. charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Comprising” or variations such as “comprise”, “comprises” or “comprised of” are used throughout the specification and claims in an inclusive sense, i.e., to specify the presence of the stated features but not to preclude the presence or addition of further features that may materially enhance the operation or utility of any of the embodiments of the invention, unless the context requires otherwise due to express language or necessary implication.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition. As a non-limiting example, if a gene signature score is defined as the composite RNA expression score for a set of genes that consists of a specified list of genes, the skilled artisan will understand that this gene signature score could include the RNA expression level determined for one or more additional genes, preferably no more than three additional genes, if such inclusion does not materially affect the predictive power.

“Framework region” or “FR” as used herein means the immunoglobulin variable regions excluding the CDR regions.

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared ×100. For example, if 8 of 10 of the positions in two sequences are matched or homologous when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, N.Y.

“Isolated antibody” and “isolated antibody fragment” refers to the purification status and in such context means the named molecule is substantially free of other biological molecules such as nucleic acids, proteins, lipids, carbohydrates, or other material such as cellular debris and growth media. Generally, the term “isolated” is not intended to refer to a complete absence of such material or to an absence of water, buffers, or salts, unless they are present in amounts that substantially interfere with experimental or therapeutic use of the binding compound as described herein.

“Kabat” as used herein means an immunoglobulin alignment and numbering system pioneered by Elvin A. Kabat ((1991) Sequences of Proteins of Immunological Interest, 5th Ed. Public Health Service, National Institutes of Health, Bethesda, Md.).

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Oligonucleotide” refers to a nucleic acid that is usually between 5 and 100 contiguous bases in length, and most frequently between 10-50, 10-40, 10-30, 10-25, 10-20, 15-50, 15-40, 15-30, 15-25, 15-20, 20-50, 20-40, 20-30 or 20-25 contiguous bases in length.

“Patient” or “subject” refers to any single subject for which therapy is desired or that is participating in a clinical trial, epidemiological study or used as a control, including humans and mammalian veterinary patients such as cattle, horses, dogs, and cats.

“PD-1 antagonist” means any chemical compound or biological molecule that blocks binding of PD-L1 expressed on a cancer cell to PD-1 expressed on an immune cell (T cell, B cell or NKT cell) and preferably also blocks binding of PD-L2 expressed on a cancer cell to the immune-cell expressed PD-1. Alternative names or synonyms for PD-1 and its ligands include: PDCD1, PD1, CD279 and SLEB2 for PD-1; PDCD1L1, PDL1, B7H1, B7-4, CD274 and B7-H for PD-L1; and PDCD1L2, PDL2, B7-DC, Btdc and CD273 for PD-L2. In any of the various aspects and embodiments of the present invention in which a human individual is being treated, the PD-1 antagonist blocks binding of human PD-L1 to human PD-1, and preferably blocks binding of both human PD-L1 and PD-L2 to human PD-1. Human PD-1 amino acid sequences can be found in NCBI Locus No.: NP_005009. Human PD-L1 and PD-L2 amino acid sequences can be found in NCBI Locus No.: NP_054862 and NP_079515, respectively.

PD-1 antagonists useful in any of the various aspects and embodiments of the present invention include a monoclonal antibody (mAb), or antigen binding fragment thereof, which specifically binds to PD-1 or PD-L1, and preferably specifically binds to human PD-1 or human PD-L1. The mAb may be a human antibody, a humanized antibody or a chimeric antibody, and may include a human constant region. In some embodiments, the human constant region is selected from the group consisting of IgG1, IgG2, IgG3 and IgG4 constant regions, and in preferred embodiments, the human constant region is an IgG1 or IgG4 constant region. In some embodiments, the antigen binding fragment is selected from the group consisting of Fab, Fab′-SH, F(ab′)₂, scFv and Fv fragments.

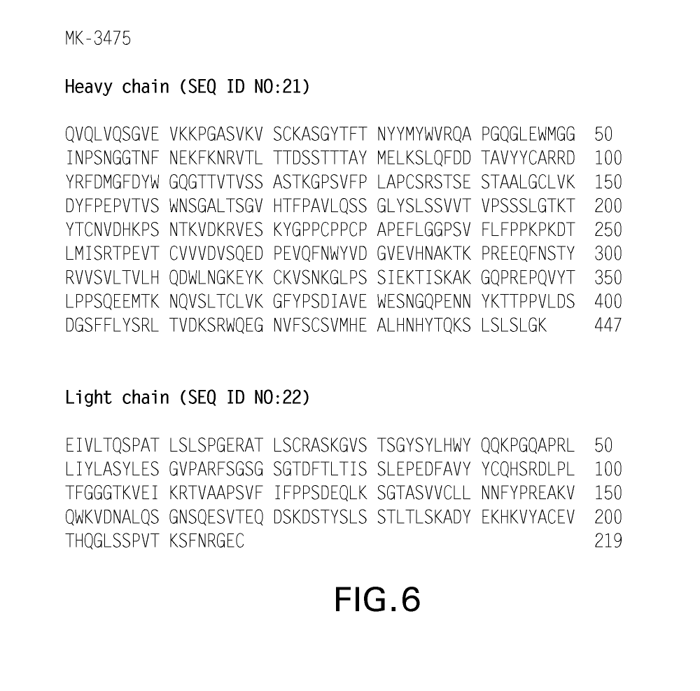

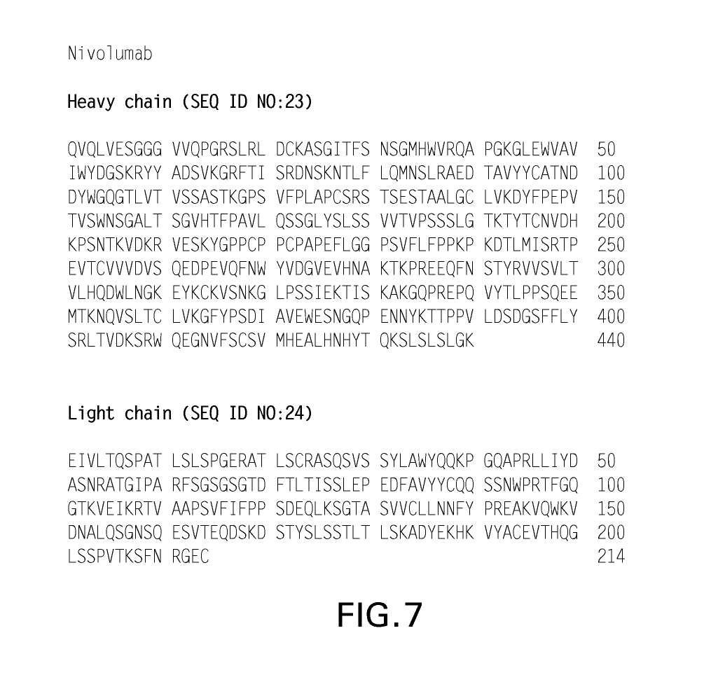

Examples of mAbs that bind to human PD-1, and useful in the various aspects and embodiments of the present invention, are described in U.S. Pat. No. 7,521,051, U.S. Pat. No. 8,008,449, and U.S. Pat. No. 8,354,509. Specific anti-human PD-1 mAbs useful as the PD-1 antagonist in the various aspects and embodiments of the present invention include: MK-3475, a humanized IgG4 mAb with the structure described in WHO Drug Information, Vol. 27, No. 2, pages 161-162 (2013) and which comprises the heavy and light chain amino acid sequences shown in FIG. 6, nivolumab (BMS-936558), a human IgG4 mAb with the structure described in WHO Drug Information, Vol. 27, No. 1, pages 68-69 (2013) and which comprises the heavy and light chain amino acid sequences shown in FIG. 7; pidilizumab (CT-011, also known as hBAT or hBAT-1); and the humanized antibodies h409A11, h409A16 and h409A17, which are described in WO2008/156712.

Examples of mAbs that bind to human PD-L1, and useful in any of the various aspects and embodiments of the present invention, are described in WO2013/019906, WO2010/077634 A1 and U.S. Pat. No. 8,383,796. Specific anti-human PD-L1 mAbs useful as the PD-1 antagonist in the various aspects and embodiments of the present invention include MPDL3280A, BMS-936559, MEDI4736, MSB0010718C and an antibody which comprises the heavy chain and light chain variable regions of SEQ ID NO:24 and SEQ ID NO:21, respectively, of WO2013/019906.

Other PD-1 antagonists useful in any of the various aspects and embodiments of the present invention include an immunoadhesin that specifically binds to PD-1 or PD-L1, and preferably specifically binds to human PD-1 or human PD-L1, e.g., a fusion protein containing the extracellular or PD-1 binding portion of PD-L1 or PD-L2 fused to a constant region such as an Fc region of an immunoglobulin molecule. Examples of immunoadhesion molecules that specifically bind to PD-1 are described in WO2010/027827 and WO2011/066342. Specific fusion proteins useful as the PD-1 antagonist in the treatment method, compositions and uses of the present invention include AMP-224 (also known as B7-DCIg), which is a PD-L2-FC fusion protein and binds to human PD-1.

In some preferred embodiments of the various aspects of the present invention, the PD-1 antagonist is a monoclonal antibody, or antigen binding fragment thereof, which comprises: (a) light chain CDRs SEQ ID NOs: 1, 2 and 3 and heavy chain CDRs SEQ ID NOs: 4, 5 and 6; or (b) light chain CDRs SEQ ID NOs: 7, 8 and 9 and heavy chain CDRs SEQ ID NOs: 10, 11 and 12.

In other preferred embodiments of the various aspects of the present invention, the PD-1 antagonist is a monoclonal antibody, or antigen binding fragment thereof, which specifically binds to human PD-1 and comprises (a) a heavy chain variable region comprising SEQ ID NO:13 or a variant thereof, and (b) a light chain variable region comprising an amino acid sequence selected from the group consisting of SEQ ID NO:15 or a variant thereof; SEQ ID NO:16 or a variant thereof; and SEQ ID NO: 17 or a variant thereof. A variant of a heavy chain variable region sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence is identical to the reference sequence except having up to five conservative amino acid substitutions in the framework region (i.e., outside of the CDRs), and preferably has less than four, three or two conservative amino acid substitution in the framework region.

In another preferred embodiment of the various aspects of the present invention, the PD-1 antagonist is a monoclonal antibody which specifically binds to human PD-1 and comprises (a) a heavy chain comprising SEQ ID NO: 14 and (b) a light chain comprising SEQ ID NO:18, SEQ ID NO:19 or SEQ ID NO:20.

In yet another preferred embodiment of the aspects of the present invention, the PD-1 antagonist is a monoclonal antibody which specifically binds to human PD-1 and comprises (a) a heavy chain comprising SEQ ID NO: 14 and (b) a light chain comprising SEQ ID NO:18.

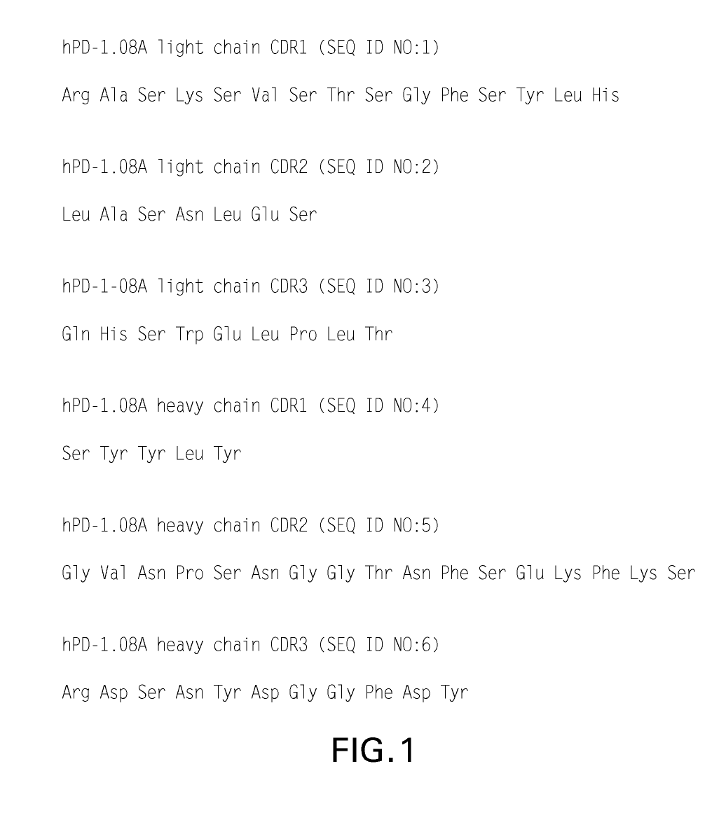

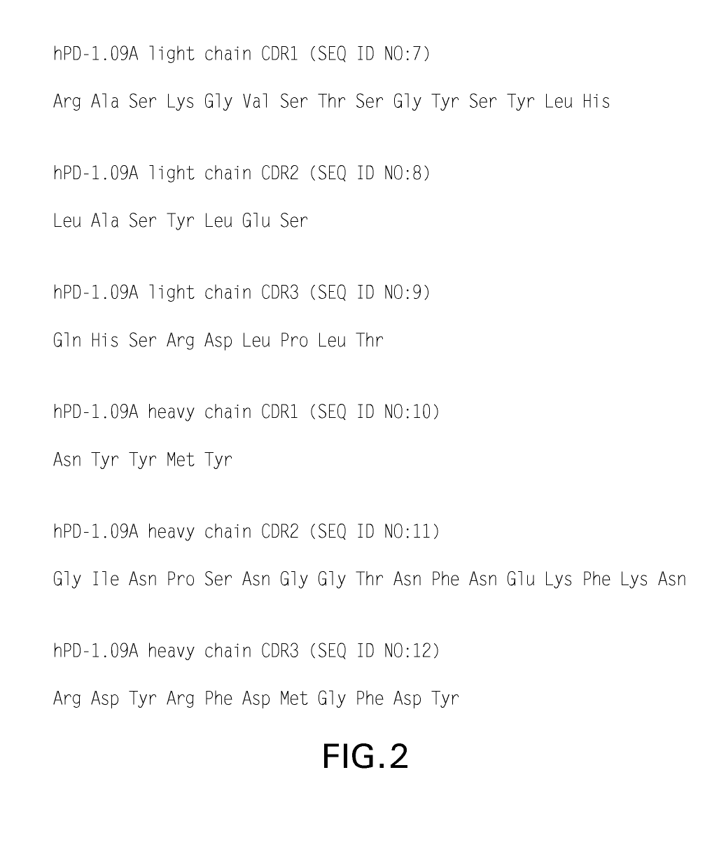

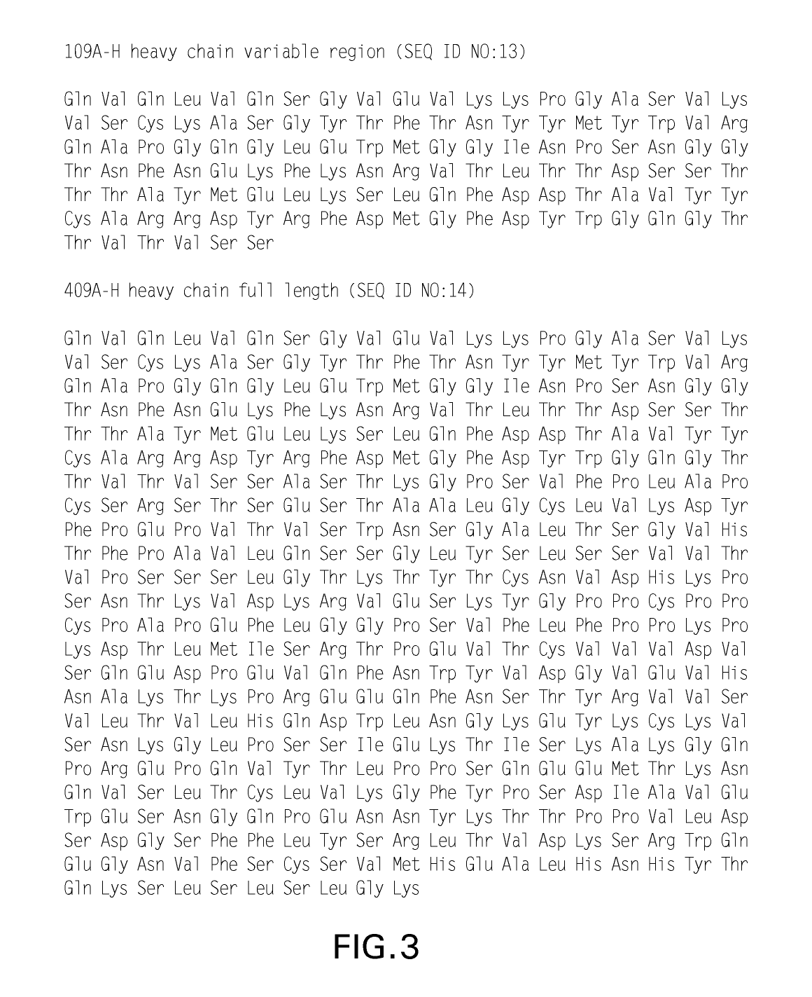

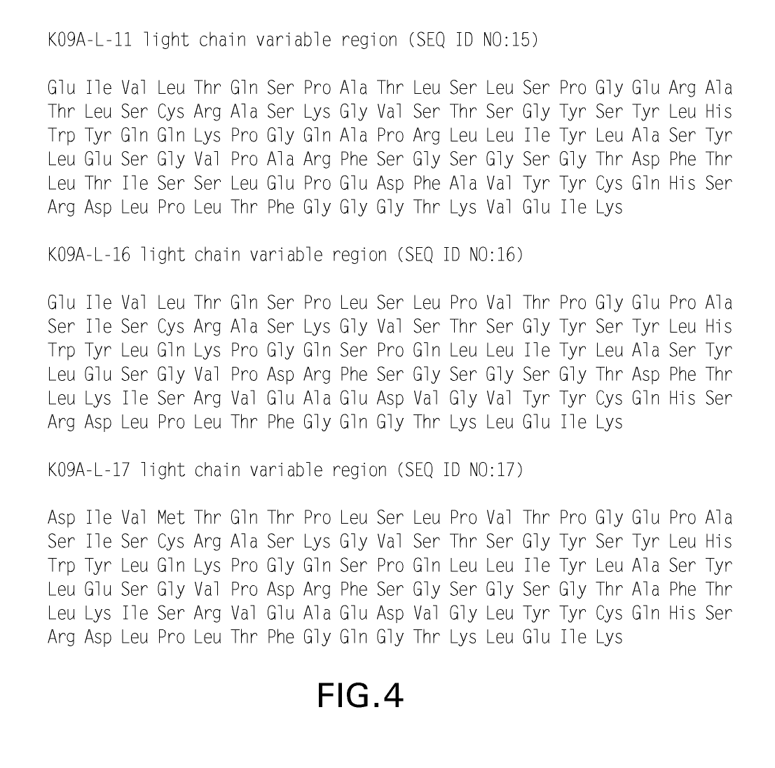

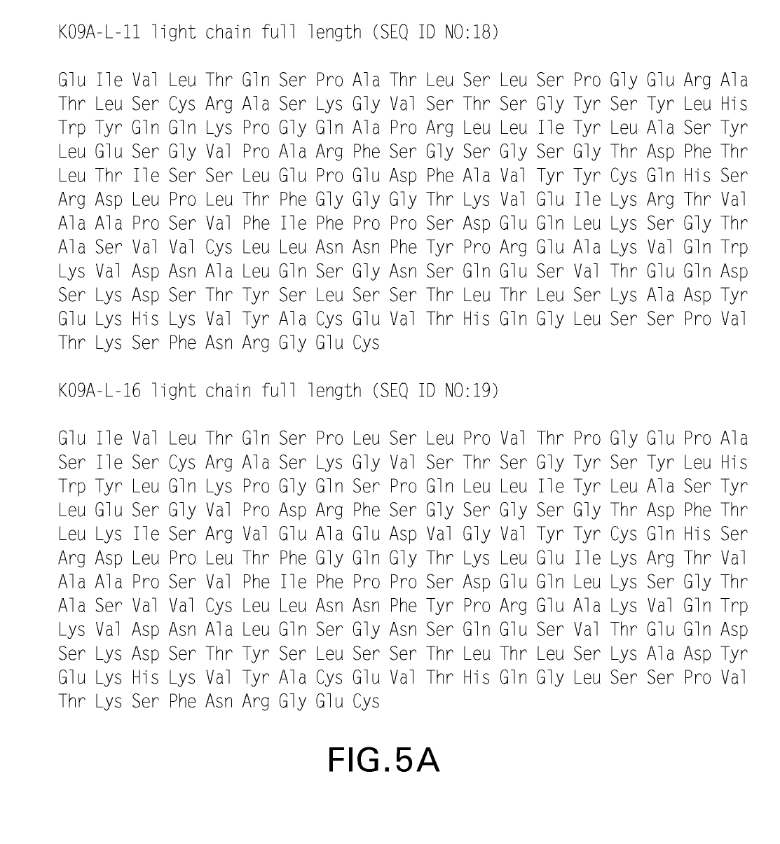

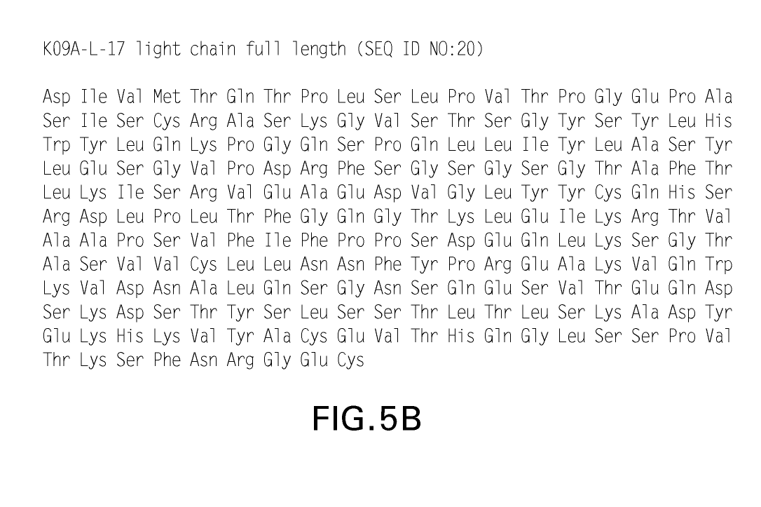

Table 3 below provides a list of the amino acid sequences of exemplary anti-PD-1 mAbs for use in the various aspects of the present invention of the present invention, and the sequences are shown in FIGS. 1-5.

TABLE 3
Exemplary anti-human PD-1 antibodies
A. Comprises light and heavy chain CDRs of
hPD-1.08A in WO2008/156712

| | |
|---|---|
| CDRL1 | SEQ ID NO: 1 |
| CDRL2 | SEQ ID NO: 2 |
| CDRL3 | SEQ ID NO: 3 |
| CDRH1 | SEQ ID NO: 4 |
| CDRH2 | SEQ ID NO: 5 |
| CDRH3 | SEQ ID NO: 6 |

B. Comprises light and heavy chain CDRs of
hPD-1.09A in WO2008/156712

| | |
|---|---|
| CDRL1 | SEQ ID NO: 7 |
| CDRL2 | SEQ ID NO: 8 |
| CDRL3 | SEQ ID NO: 9 |
| CDRH1 | SEQ ID NO: 10 |
| CDRH2 | SEQ ID NO: 11 |
| CDRH3 | SEQ ID NO: 12 |

C. Comprises the mature h109A heavy chain variable region and one of
the mature K09A light chain variable regions in WO2008/156712

| | |
|---|---|
| Heavy chain VR | SEQ ID NO: 13 |
| Light chain VR | SEQ ID NO: 15 or SEQ ID NO: 16 or SEQ ID NO: 17 |

D. Comprises the mature 409 heavy chain and one of the mature K09A
light chains in WO2008/156712

| | |
|---|---|
| Heavy chain | SEQ ID NO: 14 |
| Light chain | SEQ ID NO: 18 or SEQ ID NO: 19 or SEQ ID NO: 20 |

“Probe” as used herein means an oligonucleotide that is capable of specifically hybridizing under stringent hybridization conditions to a transcript expressed by a gene of interest listed in Table 1 or Table 4, and in some preferred embodiments, specifically hybridizes under stringent hybridization conditions to the particular transcript listed in Table 1 or Table 4 for the gene of interest.

“RECIST 1.1 Response Criteria” as used herein means the definitions set forth in Eisenhauer et al., E. A. et al., Eur. J Cancer 45:228-247 (2009) for target lesions or nontarget lesions, as appropriate based on the context in which response is being measured.

“Reference PD-L1 gene signature score” as used herein means the score for a PD-L1 gene signature that has been determined to divide at least the majority of responders from at least the majority of non-responders in a reference population of subjects who have the same tumor type as a test subject and who have been treated with a PD-1 antagonist. Preferably, at least any of 60%, 70%, 80% or 90% of responders in the reference population will have a PD-L1 gene signature score that is above the selected reference score, while the PD-L1 gene signature score for at least any of 60%, 70% 80%, 90% or 95% of the non-responders in the reference population will be lower than the selected reference score. In some embodiments, the negative predictive value of the reference score is greater than the positive predictive value. In some preferred embodiments, responders in the reference population are defined as subjects who achieved a partial response (PR) or complete response (CR) as measured by RECIST 1.1 criteria and non-responders are defined as not achieving any RECIST 1.1 clinical response. In particularly preferred embodiments, subjects in the reference population were treated with substantially the same anti-PD-1 therapy as that being considered for the test subject, i.e., administration of the same PD-1 antagonist using the same or a substantially similar dosage regimen.

“Sample” when referring to a tumor or any other biological material referenced herein, means a sample that has been removed from the subject; thus, none of the testing methods described herein are performed in or on the subject.

“Sustained response” means a sustained therapeutic effect after cessation of treatment with a therapeutic agent, or a combination therapy described herein. In some embodiments, the sustained response has a duration that is at least the same as the treatment duration, or at least 1.5, 2.0, 2.5 or 3 times longer than the treatment duration.

“Tissue Section” refers to a single part or piece of a tissue sample, e.g., a thin slice of tissue cut from a sample of a normal tissue or of a tumor.

“Treat” or “treating” a cancer as used herein means to administer a PD-1 antagonist other therapeutic agent to a subject having a cancer, or diagnosed with a cancer, to achieve at least one positive therapeutic effect, such as for example, reduced number of cancer cells, reduced tumor size, reduced rate of cancer cell infiltration into peripheral organs, or reduced rate of tumor metastasis or tumor growth. Positive therapeutic effects in cancer can be measured in a number of ways (See, W. A. Weber, J. Nucl. Med. 50:1S-10S (2009); Eisenhauer et al., supra). In some preferred embodiments, response to a PD-1 antagonist is assessed using RECIST 1.1 criteria. In some embodiments, the treatment achieved by a therapeutically effective amount is any of PR, CR, PFS, DFS, OR or OS. In some preferred embodiments, a PD-L1 gene signature biomarker of the invention predicts whether a subject with a solid tumor is likely to achieve a PR or a CR. The dosage regimen of a therapy described herein that is effective to treat a cancer patient may vary according to factors such as the disease state, age, and weight of the patient, and the ability of the therapy to elicit an anti-cancer response in the subject. While an embodiment of the treatment method, medicaments and uses of the present invention may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Tumor” as it applies to a subject diagnosed with, or suspected of having, a cancer refers to a malignant or potentially malignant neoplasm or tissue mass of any size, and includes primary tumors and secondary neoplasms. A solid tumor is an abnormal growth or mass of tissue that usually does not contain cysts or liquid areas. Different types of solid tumors are named for the type of cells that form them. Examples of solid tumors are sarcomas, carcinomas, and lymphomas. Leukemias (cancers of the blood) generally do not form solid tumors (National Cancer Institute, Dictionary of Cancer Terms).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

“Variable regions” or “V region” as used herein means the segment of IgG chains which is variable in sequence between different antibodies. It extends to Kabat residue 109 in the light chain and 113 in the heavy chain.

### II. Utility of Pd-L1 Gene Signature Biomarkers of the Invention

A PD-L1 gene signature biomarker described herein is useful to identify cancer patients who are most likely to achieve a clinical benefit from treatment with a PD-1 antagonist. This utility supports the use of these biomarkers in a variety of research and commercial applications, including but not limited to, clinical trials of PD-1 antagonists in which patients are selected on the basis of their PD-L1 gene signature score, diagnostic methods and products for determining a patient's PD-L1 gene signature score or for classifying a patient as positive or negative for a PD-L1 gene signature biomarker, personalized treatment methods which involve tailoring a patient's drug therapy based on the patient's PD-L1 gene signature score, as well as pharmaceutical compositions and drug products comprising a PD-1 antagonist for use in treating patients who test positive for a PD-L1 gene signature biomarker. The utility of any of the applications claimed herein does not require that 100% of the patients who test positive for a biomarker of the invention achieve an anti-tumor response to a PD-1 antagonist; nor does it require a diagnostic method or kit to have a specific degree of specificity or sensitivity in determining the presence or absence of a biomarker in every subject, nor does it require that a diagnostic method claimed herein be 100% accurate in predicting for every subject whether the subject is likely to have a beneficial response to a PD-1 antagonist. Thus, the inventors herein intend that the terms “determine”, “determining” and “predicting” should not be interpreted as requiring a definite or certain result; instead these terms should be construed as meaning either that a claimed method provides an accurate result for at least the majority of subjects or that the result or prediction for any given subject is more likely to be correct than incorrect.

Preferably, the accuracy of the result provided by a diagnostic method of the invention is one that a skilled artisan or regulatory authority would consider suitable for the particular application in which the method is used.
Similarly, the utility of the claimed drug products and treatment methods does not require that the claimed or desired effect is produced in every cancer patient; all that is required is that a clinical practitioner, when applying his or her professional judgment consistent with all applicable norms, decides that the chance of achieving the claimed effect of treating a given patient according to the claimed method or with the claimed composition or drug product.

### A. Testing for Biomarkers of the Invention

A PD-L1 gene signature score is determined in a sample of tumor tissue removed from a subject. The tumor may be primary or recurrent, and may be of any type (as described above), any stage (e.g., Stage I, II, III, or IV or an equivalent of other staging system), and/or histology. The subject may be of any age, gender, treatment history and/or extent and duration of remission.

The tumor sample can be obtained by a variety of procedures including, but not limited to, surgical excision, aspiration or biopsy. The tissue sample may be sectioned and assayed as a fresh specimen; alternatively, the tissue sample may be frozen for further sectioning. In some preferred embodiments, the tissue sample is preserved by fixing and embedding in paraffin or the like.

The tumor tissue sample may be fixed by conventional methodology, with the length of fixation depending on the size of the tissue sample and the fixative used. Neutral buffered formalin, glutaraldehyde, Bouin's and paraformaldehyde are nonlimiting examples of fixatives. In preferred embodiments, the tissue sample is fixed with formalin. In some embodiments, the fixed tissue sample is also embedded in paraffin to prepare an FFPE tissue sample.

Typically, the tissue sample is fixed and dehydrated through an ascending series of alcohols, infiltrated and embedded with paraffin or other sectioning media so that the tissue sample may be sectioned. Alternatively, the tumor tissue sample is first sectioned and then the individual sections are fixed.

In some preferred embodiments, the PD-L1 gene signature score for a tumor is determined using FFPE tissue sections of about 3-4 millimeters, and preferably 4 micrometers, which are mounted and dried on a microscope slide.

Once a suitable sample of tumor tissue has been obtained, it is analyzed to quantitate the expression level of each of the genes that comprise the particular PD-L1 gene signature to be scored, e.g. each of PD-L1, PD-L2, STAT1, LAG3, CXL10 and CLEC10a. The phrase “determine the expression level of a gene” as used herein refers to detecting and quantifying RNA transcribed from that gene or a protein translated from such RNA. The term “RNA transcript” includes mRNA transcribed from the gene, and/or specific spliced variants thereof and/or fragments of such mRNA and spliced variants. In preferred embodiments, the RNA transcripts whose expression is measured are the transcripts in Table 1.

A person skilled in the art will appreciate that a number of methods can be used to isolate RNA from the tissue sample for analysis. For example, RNA may be isolated from frozen tissue samples by homogenization in guanidinium isothiocyanate and acid phenol-chloroform extraction. Commercial kits are available for isolating RNA from FFPE samples.

If the tumor sample is an FFPE tissue section on a glass slide, it is preferable to perform gene expression analysis on whole cell lysates rather than on isolated total RNA. These lysates may be prepared as described in Example 1 below.

Persons skilled in the art are also aware of several methods useful for detecting and quantifying the level of RNA transcripts within the isolated RNA or whole cell lysates. Quantitative detection methods include, but are not limited to, arrays (i.e., microarrays), quantitative real time PCR (RT-PCR), multiplex assays, nuclease protection assays, and Northern blot analyses. Generally, such methods employ labeled probes that are complimentary to a portion of each transcript to be detected. Probes for use in these methods can be readily designed based on the known sequences of the genes and the transcripts expressed thereby. In some preferred embodiments, the probes are designed to hybridize to each of the gene signature transcripts identified in Table 1 for PD-L1, PD-L2, STAT1, LAG3, CXCL10 and CLEC10a. Suitable labels for the probes are well-known and include, e.g., fluorescent, chemiluminescent and radioactive labels.

In some embodiments, assaying a tumor sample for a PD-L1 gene signature employs detection and quantification of RNA levels in real-time using nucleic acid sequence based amplification (NASBA) combined with molecular beacon detection molecules. NASBA is described, e.g., in Compton J., Nature 350 (6313):91-92 (1991). NASBA is a single-step isothermal RNA-specific amplification method. Generally, the method involves the following steps: RNA template is provided to a reaction mixture, where the first primer attaches to its complementary site at the 3′ end of the template; reverse transcriptase synthesizes the opposite, complementary DNA strand; RNAse H destroys the RNA template (RNAse H only destroys RNA in RNA-DNA hybrids, but not single-stranded RNA); the second primer attaches to the 3′ end of the DNA strand, and reverse transcriptase synthesizes the second strand of DNA; and T7 RNA polymerase binds double-stranded DNA and produces a complementary RNA strand which can be used again in step 1, such that the reaction is cyclic.

In other embodiments, the assay format is a flap endonuclease-based format, such as the Invader™ assay (Third Wave Technologies). In the case of using the invader method, an invader probe containing a sequence specific to the region 3′ to a target site, and a primary probe containing a sequence specific to the region 5′ to the target site of a template and an unrelated flap sequence, are prepared. Cleavase is then allowed to act in the presence of these probes, the target molecule, as well as a FRET probe containing a sequence complementary to the flap sequence and an auto-complementary sequence that is labeled with both a fluorescent dye and a quencher. When the primary probe hybridizes with the template, the 3′ end of the invader probe penetrates the target site, and this structure is cleaved by the Cleavase resulting in dissociation of the flap. The flap binds to the FRET probe and the fluorescent dye portion is cleaved by the Cleavase resulting in emission of fluorescence.

In yet other embodiments, the assay format employs direct mRNA capture with branched DNA (QuantiGene™, Panomics) or Hybrid Capture™ (Digene).

One example of an array technology suitable for use in measuring expression of the genes in a PD-L1 gene signature is the ArrayPlate™ assay technology sold by HTG Molecular, Tucson Ariz., and described in Martel, R. R., et al., Assay and Drug Development Technologies 1(1):61-71, 2002. In brief, this technology combines a nuclease protection assay with array detection. Cells in microplate wells are subjected to a nuclease protection assay. Cells are lysed in the presence of probes that bind targeted mRNA species. Upon addition of SI nuclease, excess probes and unhybridized mRNA are degraded, so that only mRNA:probe duplexes remain. Alkaline hydrolysis destroys the mRNA component of the duplexes, leaving probes intact. After the addition of a neutralization solution, the contents of the processed cell culture plate are transferred to another ArrayPlate™ called a programmed ArrayPlate™. ArrayPlates™ contain a 16-element array at the bottom of each well. Each array element comprises a position-specific anchor oligonucleotide that remains the same from one assay to the next. The binding specificity of each of the 16 anchors is modified with an oligonucleotide, called a programming linker oligonucleotide, which is complementary at one end to an anchor and at the other end to a nuclease protection probe. During a hybridization reaction, probes transferred from the culture plate are captured by immobilized programming linker. Captured probes are labeled by hybridization with a detection linker oligonucleotide, which is in turn labeled with a detection conjugate that incorporates peroxidase. The enzyme is supplied with a chemiluminescent substrate, and the enzyme-produced light is captured in a digital image. Light intensity at an array element is a measure of the amount of corresponding target mRNA present in the original cells.

By way of further example, DNA microarrays can be used to measure gene expression. In brief, a DNA microarray, also referred to as a DNA chip, is a microscopic array of DNA fragments, such as synthetic oligonucleotides, disposed in a defined pattern on a solid support, wherein they are amenable to analysis by standard hybridization methods (see Schena, BioEssays 18:427 (1996)). Exemplary microarrays and methods for their manufacture and use are set forth in T. R. Hughes et al., Nature Biotechnology 9:342-347 (2001). A number of different microarray configurations and methods for their production are known to those of skill in the art and are disclosed in U.S. Pat. Nos. 5,242,974; 5,384,261; 5,405,783; 5,412,087; 5,424,186; 5,429,807; 5,436,327; 5,445,934; 5,556,752; 5,405,783; 5,412,087; 5,424,186; 5,429,807; 5,436,327; 5,472,672; 5,527,681; 5,529,756; 5,545,531; 5,554,501; 5,561,071; 5,571,639; 5,593,839; 5,624,711; 5,700,637; 5,744,305; 5,770,456; 5,770,722; 5,837,832; 5,856,101; 5,874,219; 5,885,837; 5,919,523; 6,022,963; 6,077,674; and U.S. Pat. No. 6,156,501; Shena, et al., Tibtech 6:301-306, 1998; Duggan, et al., Nat. Genet. 2:10-14, 1999; Bowtell, et al., Nat. Genet. 21:25-32, 1999; Lipshutz, et al., Nat. Genet. 21:20-24, 1999; Blanchard, et al., Biosensors and Bioelectronics 77:687-90, 1996; Maskos, et al., Nucleic Acids Res. 2:4663-69, 1993; and Hughes, et al., Nat. Biotechnol. 79:342-347, 2001. Patents describing methods of using arrays in various applications include: U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,848,659; and 5,874,219; the disclosures of which are herein incorporated by reference.

In one embodiment, an array of oligonucleotides may be synthesized on a solid support. Exemplary solid supports include glass, plastics, polymers, metals, metalloids, ceramics, organics, etc. Using chip masking technologies and photoprotective chemistry, it is possible to generate ordered arrays of nucleic acid probes. These arrays, which are known, for example, as “DNA chips” or very large scale immobilized polymer arrays (“VLSIPS®” arrays), may include millions of defined probe regions on a substrate having an area of about 1 cm² to several cm², thereby incorporating from a few to millions of probes (see, e.g., U.S. Pat. No. 5,631,734).

To compare expression levels, labeled nucleic acids may be contacted with the array under conditions sufficient for binding between the target nucleic acid and the probe on the array. In one embodiment, the hybridization conditions may be selected to provide for the desired level of hybridization specificity; that is, conditions sufficient for hybridization to occur between the labeled nucleic acids and probes on the microarray.

Hybridization may be carried out in conditions permitting essentially specific hybridization. The length and GC content of the nucleic acid will determine the thermal melting point and thus, the hybridization conditions necessary for obtaining specific hybridization of the probe to the target nucleic acid. These factors are well known to a person of skill in the art, and may also be tested in assays. An extensive guide to nucleic acid hybridization may be found in Tijssen, et al. (Laboratory Techniques in Biochemistry and Molecular Biology, Vol. 24: Hybridization With Nucleic Acid Probes, P. Tijssen, ed.; Elsevier, N.Y. (1993)). The methods described above will result in the production of hybridization patterns of labeled target nucleic acids on the array surface. The resultant hybridization patterns of labeled nucleic acids may be visualized or detected in a variety of ways, with the particular manner of detection selected based on the particular label of the target nucleic acid. Representative detection means include scintillation counting, autoradiography, fluorescence measurement, calorimetric measurement, light emission measurement, light scattering, and the like.

One such method of detection utilizes an array scanner that is commercially available (Affymetrix, Santa Clara, Calif.), for example, the 417® Arrayer, the 418® Array Scanner, or the Agilent Gene Array® Scanner. This scanner is controlled from a system computer with an interface and easy-to-use software tools. The output may be directly imported into or directly read by a variety of software applications. Exemplary scanning devices are described in, for example, U.S. Pat. Nos. 5,143,854 and 5,424,186.

A preferred assay method to measure biomarker transcript abundance includes using the nCounter® Analysis System marketed by NanoString® Technologies (Seattle, Wash. USA). This system, which is described by Geiss et al., Nature Biotechnol. 2(3):317-325 (2008), utilizes a pair of probes, namely, a capture probe and a reporter probe, each comprising a 35- to 50-base sequence complementary to the transcript to be detected. The capture probe additionally includes a short common sequence coupled to an immobilization tag, e.g. an affinity tag that allows the complex to be immobilized for data collection. The reporter probe additionally includes a detectable signal or label, e.g. is coupled to a color-coded tag. Following hybridization, excess probes are removed from the sample, and hybridized probe/target complexes are aligned and immobilized via the affinity or other tag in a cartridge. The samples are then analyzed, for example using a digital analyzer or other processor adapted for this purpose. Generally, the color-coded tag on each transcript is counted and tabulated for each target transcript to yield the expression level of each transcript in the sample. This system allows measuring the expression of hundreds of unique gene transcripts in a single multiplex assay using capture and reporter probes designed by NanoString.

In measuring expression of the genes in a PD-L1 gene signature disclosed herein, the absolute expression of each of the genes in a tumor sample is compared to a control; for example, the control can be the average level of expression of each of the genes, respectively, in a pool of subjects. To increase the sensitivity of the comparison, however, the expression level values are preferably transformed in a number of ways.

For example, the expression level of each gene in the gene signature can be normalized by the average expression level of all of the genes, the expression level of which is determined, or by the average expression level of a set of control genes. Thus, in one embodiment, the genes are represented by a set of probes, and the expression level of each of the genes is normalized by the mean or median expression level across all of the genes represented, including any genes that are not part of the gene signature of interest. In a specific embodiment, the normalization is carried out by dividing the median or mean level of expression of all of the genes on the microarray. In another embodiment, the expression levels of the signature genes are normalized by the mean or median level of expression of a set of control genes. In a specific embodiment, the control genes comprise housekeeping genes. In another specific embodiment, the normalization is accomplished by dividing by the median or mean expression level of the control genes.

The sensitivity of a gene signature score will also be increased if the expression levels of individual genes in the gene signature are compared to the expression of the same genes in a pool of tumor samples. Preferably, the comparison is to the mean or median expression level of each signature gene in the pool of samples. Such a comparison may be accomplished, for example, by dividing by the mean or median expression level of the pool for each of the genes from the expression level each of the genes in the subject sample of interest. This has the effect of accentuating the relative differences in expression between genes in the sample and genes in the pool as a whole, making comparisons more sensitive and more likely to produce meaningful results than the use of absolute expression levels alone. The expression level data may be transformed in any convenient way; preferably, the expression level data for all is log transformed before means or medians are taken.

In performing comparisons to a pool, two approaches may be used. First, the expression levels of the signature genes in the sample may be compared to the expression level of those genes in the pool, where nucleic acid derived from the sample and nucleic acid derived from the pool are hybridized during the course of a single experiment. Such an approach requires that a new pool of nucleic acid be generated for each comparison or limited numbers of comparisons, and is therefore limited by the amount of nucleic acid available. Alternatively, and preferably, the expression levels in a pool, whether normalized and/or transformed or not, are stored on a computer, or on computer-readable media, to be used in comparisons to the individual expression level data from the sample (i.e., single-channel data).

When comparing a subject's tumor sample with a standard or control, the expression value of a particular gene in the sample is compared to the expression value of that gene in the standard or control. For each gene in a PD-L1 gene signature, the log(10) ratio is created for the expression value in the individual sample relative to the standard or control. A score for a PD-L1 gene signature is calculated by determining the mean log(10) ratio of the genes in the signature. If the gene signature score for the test sample is at or above a pre-determined threshold, then the sample is considered to be positive for a PD-L1 gene signature biomarker. In one embodiment of the invention, the pre-determined threshold is set at any number between 1.80 and 2.40 (i.e., 1.81, 1.82, 1.83 . . . 2.37, 2.38, 2.39). The pre-determined threshold may also be the mean, median, or a percentile of scores for that PD-L1 gene signature in a collection of samples or a pooled sample used as a standard or control.

It will be recognized by those skilled in the art that other differential expression values, besides log(10) ratio, may be used for calculating a signature score, as long as the value represents an objective measurement of transcript abundance of the genes. Examples include, but are not limited to: xdev, error-weighted log (ratio), and mean subtracted log(intensity).

In one preferred embodiment, raw expression values are normalized by performing quantile normalization relative to the reference distribution and subsequent log 10-transformation. When the gene expression is detected using the nCounter® Analysis System marketed by NanoString® Technologies, the reference distribution is generated by pooling reported (i.e., raw) counts for the test sample and one or more control samples (preferably at least 2 samples, more preferably at least 4, 8 or 16 samples) after excluding values for technical (both positive and negative control) probes and without performing intermediate normalization relying on negative (background-adjusted) or positive (synthetic sequences spiked with known titrations). The PD-L1 signature score is then calculated as the arithmetic mean of normalized values for each of the genes in the gene signature, e.g., PD-L1, PD-L2, STAT1, LAG3, CXCL10, and CLEC10a.

In some preferred embodiments, the reference distribution is generated from raw expression counts for a normalization set of genes, which consists essentially of each of the genes in the set of 400 genes listed in Table 4, or a subset thereof. The subset may consist of at least any of 25, 50, 75, 100, 125, 150, 175, 200, 225, 250, 275, 300, 325, 350, 375 or any whole number in between 25 and 400.

TABLE 4
Normalization Gene Set

| | Target Transcript |
|---|---|
| Gene Id | NCBI Accession # |
| ABCF1 | NM_001090.2 |
| ALAS1 | NM_000688.4 |
| AXL | NM_021913.2 |
| Adipoq | NM_004797.2 |
| Areg | NM_001657.2 |
| Arg1 | NM_000045.2 |
| Arg2 | NM_001172.3 |
| Atp6v0d2 | NM_152565.1 |
| Atp8b4 | NM_024837.2 |
| B7-H3 (CD276) | NM_001024736.1 |
| B7-H4 (VTCN1) | NM_024626.2 |
| BAGE | NM_001187.1 |
| BCL6 | NM_138931.1 |
| BLNK | NM_013314.2 |
| Batf | NM_006399.3 |
| Bcl11a | NM_022893.3 |
| Bcl11b | NM_022898.1 |
| Bst1 | NM_004334.2 |
| Btla | NM_181780.2 |
| CADM1 | NM_014333.3 |
| CD112 | NM_002856.2 |
| CD113 | NM_015480.2 |
| CD127 (IL-7RA) | NM_002185.2 |
| CD14 | NM_000591.2 |
| CD155 | NM_006505.3 |
| CD160 | NM_007053.2 |
| CD163 | NM_004244.4 |
| CD167 DDR1 | NM_001954.4 |
| CD2 | NM_001767.2 |
| CD200 | NM_005944.5 |
| CD200R1 | NM_138939.2 |
| CD207-CLEC4K | NM_015717.2 |
| Langerin | |
| CD209 | NM_021155.2 |
| CD22 (Siglec-2) | NM_001771.2 |
| CD226 | NM_006566.2 |
| CD244 | NM_016382.2 |
| CD24a | NM_013230.2 |
| CD28 | NM_001243078.1 |
| CD3 delta | NM_000732.4 |
| CD3 epsilon | NM_000733.2 |
| CD3 zeta (CD247) | NM_198053.1 |
| CD300a | NM_007261.2 |
| CD300b (CD300LB | NM_174892.2 |
| IREM3) | |
| CD300e (IREM2) | NM_181449.1 |
| CD300f (IREM1) | NM_139018.3 |
| CD317 (Bst2) | NM_004335.2 |
| CD33 | NM_001177608.1 |
| CD4 | NM_000616.3 |
| CD40 (TNFRSF5) | NM_001250.4 |
| CD40L (TNFSF5) | NM_000074.2 |
| CD44 | NM_001001392.1 |
| CD45 (PTPRC) | NM_080921.2 |
| CD47 | NM_001777.3 |
| CD48 | NM_001778.2 |
| CD5 | NM_014207.2 |
| CD55 | NM_000574.3 |
| CD62L L-selectin | NR_029467.1 |
| Sell | |
| CD68 (SCARD1) | NM_001251.2 |
| CD69 | NM_001781.1 |
| CD7 | NM_006137.6 |
| CD72 | NM_001782.2 |
| CD79A | NM_001783.3 |
| CD80 | NM_005191.3 |
| CD84 | NM_001184879.1 |
| CD86 | NM_175862.3 |
| CD8b | NM_172099.2 |
| CD90 (Thy1) | NM_006288.2 |
| CD96 | NM_005816.4 |
| CDH1 (E Cadherin) | NM_004360.2 |
| CLEC12A | NM_138337.5 |
| CLEC15a (KLRG1 | NM_005810.3 |
| MAFA) | |
| CLEC4A | NM_194448.2 |
| CLEC6A | NM_001007033.1 |
| CSPG4 | NM_001897.4 |
| CXCL11-ITAC | NM_005409.3 |
| CXCL2 (GRO-beta | NM_002089.3 |
| MIP-2) | |
| CXCL9-Mig | NM_002416.1 |
| CXCR2 | NM_001557.2 |
| Caspase 3 | NM_032991.2 |
| Ccl19 | NM_006274.2 |
| Ccl21 | NM_002989.2 |
| Ccl24 | NM_002991.2 |
| Ccl27 | NM_006664.2 |
| Ccl3 | NM_002983.2 |
| Ccl4 | NM_002984.2 |
| Ccl5 | NM_002985.2 |
| Ccl8 | NM_005623.2 |
| Ccr2 | NM_001123041.2 |
| Ccr3 | NM_001837.2 |
| Ccr4 | NM_005508.4 |
| Ccr5 | NM_000579.1 |
| Ccr6 | NM_031409.2 |
| Ccr7 | NM_001838.2 |
| Cdo1 | NM_001801.2 |
| Chi3l1 | NM_001276.2 |
| Chi3l2 | NM_004000.2 |
| Ciita | NM_000246.3 |
| Clca1 | NM_001285.3 |
| Clca2 | NM_006536.5 |
| Clec10a (mouse also | NM_182906.2 |
| MGL1) | |
| Clec1b (Clec-2) | NM_016509.3 |
| Clec2d (OCIL) | NM_001004419.3 |
| Clec3b | NM_003278.2 |
| Clec4d (MCL) | NM_080387.4 |
| Clec4e (Mincle) | NM_014358.2 |
| Clec5a (MDL-1) | NM_013252.2 |
| Clec7a (dectin-1) | NM_197954.2 |
| Clec9a | NM_207345.2 |
| Cmklr1 | NM_004072.1 |
| Cpd | NM_001304.4 |
| Crtam | NM_019604.2 |
| Csf1r | NM_005211.2 |
| Csf2rb | NM_000395.2 |
| Cst6 | NM_001323.3 |
| Cst7 | NM_003650.3 |
| Ctla4 | NM_005214.3 |
| Ctsb | NM_000100.2 |
| Ctsg | NM_001911.2 |
| Ctsz | NM_001336.3 |
| Cx3cl1 | NM_002996.3 |
| Cx3cr1 | NM_001337.3 |
| Cxcl1 (GRO-alpha) | NM_001511.1 |
| Cxcl10 (IP-10) | NM_001565.1 |
| Cxcl13 (BCA-1) | NM_006419.2 |
| Cxcl14 | NM_004887.4 |
| Cxcl3 | NM_002090.2 |
| Cxcl4 (Pf4) | NM_002619.2 |
| Cxcr3 | NM_001504.1 |
| Cxcr6 | NM_006564.1 |
| Cxcr7 | NM_020311.1 |
| DCK | NM_000788.2 |
| DCT | NM_001922.3 |
| Dab1 | NM_021080.3 |
| Dap10 (HCST) | NM_001007469.1 |
| Dap12 (TYROBP) | NM_003332.2 |
| Def6 | NM_022047.3 |
| Defb1 | NM_005218.3 |
| Defb2 | NM_004942.2 |
| Dgkz | NM_001105540.1 |
| Dpp4 (CD26) | NM_001935.3 |
| Dsc1 | NM_024421.2 |
| Dsc2 | NM_024422.3 |
| Dsg2 | NM_001943.3 |
| EEF1G | NM_001404.4 |
| EGF | NM_001963.3 |
| Efemp1 | NM_004105.3 |
| Egfr | NM_201282.1 |
| Egr2 | NM_000399.3 |
| Eomes | NM_005442.2 |
| Epcam | NM_002354.1 |
| Ezr | NM_003379.4 |
| F2R (PAR-1) | NM_001992.2 |
| F2RL1 (PAR-2) | NM_005242.3 |
| FCER1A | NM_002001.2 |
| FCGR2A (CD32) | NM_021642.2 |
| FN1 | NM_212482.1 |
| Fap | NM_004460.2 |
| Fasl (TNFSF6) | NM_000639.1 |
| Fcgr2b (CD32b) | NM_001002273.1 |
| Fcrl3 | NM_052939.3 |
| Folr4 | NM_001199206.1 |
| Foxp3 | NM_014009.3 |
| G6PD | NM_000402.2 |
| GAPDH | NM_002046.3 |
| GUSB | NM_000181.1 |
| Gas6 | NM_000820.2 |
| Gata3 | NM_001002295.1 |
| Gdf10 | NM_004962.2 |
| Gfi1 | NM_005263.2 |
| Gitr (Tnfrsf18) | NM_004195.2 |
| Gitrl (Tnfsf18) | NM_005092.2 |
| Gnly | NM_006433.2 |
| Gpld1 | NM_001503.2 |
| gpr18 | NM_001098200.1 |
| Grap2 | NM_004810.2 |
| Gzma | NM_006144.2 |
| Gzmb | NM_004131.3 |
| Gzmk | NM_002104.2 |
| HLA-A (HLA Class | NM_002116.5 |
| I) | |
| HLA-B | NM_005514.6 |
| HLA-C | NM_002117.4 |
| HLA-DRA (HLA | NM_019111.3 |
| class II) | |
| HLA-E | NM_005516.4 |
| HPRT1 | NM_000194.1 |
| Havcr1-Tim1 | NM_001099414.1 |
| Havcr2-Tim3 | NM_032782.3 |
| Hcls1 | NM_005335.4 |
| Hgfac | NM_001528.2 |
| Hif1a | NM_001530.2 |
| Hopx | NM_001145460.1 |
| IFNg | NM_000619.2 |
| IGSF6 | NM_005849.2 |
| IL-10R1 | NM_001558.2 |
| IL-2RA | NM_000417.1 |
| IL-2RB | NM_000878.2 |
| IL-2Rg | NM_000206.1 |
| IL-37 | NM_014439.3 |
| IL10 | NM_000572.2 |
| IL18 | NM_001562.2 |
| IL18R1 | NM_003855.2 |
| IL2 | NM_000586.2 |
| IL4 | NM_000589.2 |
| ITGAL (CD11a) | NM_002209.2 |
| ITGAM (CD11b) | NM_000632.3 |
| Icam1 | NM_000201.1 |
| Icos | NM_012092.2 |
| IcosL (B7-H2) | NM_015259.4 |
| Id2 | NM_002166.4 |
| Ido1 (Indo) | NM_002164.3 |
| Ifi16 | NM_005531.1 |
| Ifitm1 | NM_003641.3 |
| Ifngr2 | NM_005534.3 |
| Igf1 | NM_000618.3 |
| Igj | NM_144646.3 |
| Ikzf3 | NM_012481.3 |
| Ing1 | NM_198219.1 |
| Ing2 | NM_001564.2 |
| Insr | NM_000208.1 |
| Irf1 | NM_002198.1 |
| Irf2 | NM_002199.2 |
| Irf4 | NM_002460.1 |
| Irf6 | NM_006147.2 |
| Irf7 | NM_001572.3 |
| Irf8 | NM_002163.2 |
| Itga1 (CD49) | NM_181501.1 |
| Itga2 (CD49b) | NM_002203.2 |
| Itgae (CD103) | NM_002208.4 |
| Itgax | NM_000887.3 |
| Itk | NM_005546.3 |
| Itm2a | NM_004867.4 |
| Jak3 | NM_000215.2 |
| Jakmip1 | NM_001099433.1 |
| KIR2DL1 | NM_014218.2 |
| KLK6 | NM_002774.3 |
| KLRG2 (CLEC15b) | NM_198508.2 |
| Klrc1 (NKG2A) | NM_002259.3 |
| Klrc2 (NKG2c) | NM_002260.3 |
| Klrd1 (CD94) | NM_002262.3 |
| Klrk1-NKG2D | NM_007360.1 |
| LAIR1 | NM_002287.3 |
| LIFR | NM_002310.3 |
| LILRA1 (CD85I) | NM_006863.1 |
| LILRA2 v1-2 | NM_001130917.1 |
| (CD85H) | |
| LILRA4 (CD85G) | NM_012276.3 |
| LILRA5 v3-4 | NM_181879.1 |
| (CD85F) | |
| Lag3 (CD223) | NM_002286.5 |
| Lamp2 | NM_002294.2 |
| Lat | NM_001014987.1 |
| Lat2-linker for | NM_014146.3 |
| activation of T cells | |
| family member 2 | |
| Lax1 | NM_001136190.1 |
| Lck | NM_005356.2 |
| Lgals3 | NM_001177388.1 |
| Lgals3BP | NM_005567.3 |
| Lgals9-lectin | NM_002308.3 |
| LilRB4 | NM_001081438.1 |
| Lst1 | NM_001166538.1 |
| Ltk | NM_002344.5 |
| Ly6e | NM_002346.2 |
| Ly6g6c | NM_025261.2 |
| Ly6g6d | NM_021246.2 |
| MAGEA1-melanoma | NM_004988.4 |
| antigen family A | |
| MBL2 | NM_000242.2 |
| MER (MERTK) | NM_006343.2 |
| MLANA (Mart1) | NM_005511.1 |
| MON1B | NM_014940.2 |
| MSA41 (CD20) | NM_152866.2 |
| Maf | NM_001031804.2 |
| Mafb | NM_005461.3 |
| Marco (Scara2) | NM_006770.3 |
| Mica | NM_000247.1 |
| Micb | NM_005931.3 |
| Mn1 | NM_002430.2 |
| Mrc1 | NM_002438.2 |
| Myh4 | NM_017533.2 |
| NCR2-NKp44 | NM_004828.3 |
| Nfatc1 | NM_172389.1 |
| Nkg7 | NM_005601.3 |
| Nlrp10 (NOD) | NM_176821.3 |
| Nr4a2 | NM_006186.3 |
| Ny-eso-1 (CTAG1B) | NM_001327.2 |
| OAZ1 | NM_004152.2 |
| OSCAR | NM_130771.3 |
| PARK7 | NM_001123377.1 |
| PD-1 (Pdcd1) | NM_005018.1 |
| PDCD4 | NM_014456.3 |
| POLR1B | NM_019014.3 |
| POLR2A | NM_000937.2 |
| PPARG | NM_015869.3 |
| PPIA | NM_021130.2 |
| Pdcd1Lg1 (PD-L1) | NM_014143.2 |
| Pdcd1Lg2 (PD-L2) | NM_025239.3 |
| Pdgfra | NM_006206.3 |
| Phactr2 | NM_001100164.1 |
| Pi3kCA | NM_006218.2 |
| Pi3kCB | NM_006219.1 |
| Pi3kCD | NM_005026.3 |
| Pi3kCG | NM_002649.2 |
| Pilra (FDF03 | NM_178273.1 |
| inhibited) | |
| Pilrb (FDF03 | NM_178238.1 |
| activated) | |
| Postn | NM_001135935.1 |
| Ppp1r2 | NM_006241.4 |
| Prf1 | NM_005041.3 |
| Psmb10 | NM_002801.2 |
| Psmb8 | NM_004159.4 |
| Psmb9 | NM_002800.4 |
| Psme1 | NM_006263.2 |
| Psme2 | NM_002818.2 |
| Pstpip1 | NM_003978.3 |
| Pstpip2 | NM_024430.3 |
| Pten | NM_000314.3 |
| Ptger2 | NM_000956.2 |
| Ptger4 | NM_000958.2 |
| Ptpn10 (Dusp1) | NM_004417.2 |
| Ptpn13 | NM_080684.2 |
| Ptpn22 | NM_015967.3 |
| Ptpn3 | NM_001145372.1 |
| Ptpn6 | NM_002831.5 |
| Ptpn7 | NM_002832.3 |
| Ptprcap | NM_005608.2 |
| Ptprf | NM_002840.3 |
| Pvrig | NM_024070.3 |
| RGS16 | NM_002928.2 |
| RIKEN cDNA | NM_022153.1 |
| 4632428N05 | |
| (VISTA) | |
| RPL19 | NM_000981.3 |
| Rarres2 | NM_002889.3 |
| Retnlb (Relmb Fizz2) | NM_032579.2 |
| Rgn | NM_152869.2 |
| Rora | NM_134261.2 |
| Rorc (RORg and T) | NM_001001523.1 |
| Runx1 | NM_001754.4 |
| Runx3 | NM_004350.1 |
| S100a8 | NM_002964.3 |
| S100a9 | NM_002965.2 |
| SAMD3 | NM_001017373.2 |
| SART3 | NM_014706.3 |
| SDHA | NM_004168.1 |
| SIGLEC14 | NM_001098612.1 |
| SIGLEC15 | NM_213602.2 |
| (CD33L3) | |
| SIGLEC5 (CD170; | NM_003830.2 |
| CD33L2) | |
| Samhd1 | NM_015474.2 |
| Sema4a | NM_001193300.1 |
| Serpinf1 | NM_002615.4 |
| Sgpp2 | NM_152386.2 |
| Sh2d1b | NM_053282.4 |
| Sh2d2a | NM_001161443.1 |
| Sirpb1 | NM_006065.3 |
| Sirpg | NM_001039508.1 |
| Sit1 | NM_014450.2 |
| Sla1 | NM_001045556.2 |
| Sla2 | NM_032214.2 |
| Slamf1 (CD150 | NM_003037.2 |
| Slam) | |
| Slamf6 (ntba) | NM_001184714.1 |
| Slamf7 (Cracc) | NM_021181.3 |
| Socs3 | NM_003955.3 |
| Stat1 | NM_007315.2 |
| Stat6 | NM_003153.3 |
| TBP | NM_001172085.1 |
| TIMP3 | NM_000362.4 |
| TIMP4 | NM_003256.2 |
| TNFRSF10b- | NM_003842.3 |
| TRAIL R2 DR5 | |
| TNFRSF13B-TACI | NM_012452.2 |
| TNFRSF8-CD30 | NM_152942.2 |
| TNFSF10-TRAIL | NM_003810.2 |
| CD253 | |
| TNFSF13b-BLYS | NM_006573.4 |
| TNFSF8-CD30L | NM_001244.2 |
| TREM1 | NM_018643.3 |
| TREM2 | NM_018965.2 |
| TREML1 (TLT-1) | NM_178174.2 |
| TREML2 (TLT-2) | NM_024807.2 |
| TUBB | NM_178014.2 |
| TYR (Tyrosinase) | NM_000372.4 |
| TYRO3 | NM_006293.2 |
| Tagap | NM_054114.3 |
| Tarp (TCR gamma | NM_001003799.1 |
| alternate reading | |
| frame protein) | |
| Tbx21 (Tbet) | NM_013351.1 |
| Tcn2 | NM_000355.2 |
| Tigit | NM_173799.2 |
| Tmem2 | NM_013390.2 |
| Tnfa | NM_000594.2 |
| Tnfaip3 | NM_006290.2 |
| Tnfaip6 | NM_007115.2 |
| Tnfaip8L2 | NM_024575.3 |
| Tnfrsf14 (Hvem) | NM_003820.2 |
| Tnfrsf4 (Ox40) | NM_003327.2 |
| Tnfrsf7 (Cd27) | NM_001242.4 |
| Tnfrsf9 (CD137 4- | NM_001561.4 |
| 1BB) | |
| Tnfsf14 (LIGHT) | NM_003807.2 |
| Tnfsf4 | NM_003326.2 |
| Tnfsf7 CD27L | NM_001252.2 |
| Tnfsf9 (4-1BBL) | NM_003811.3 |
| Tox | NM_014729.2 |
| Trat1 | NM_016388.2 |
| UBB | NM_018955.2 |
| Ubash3a | NM_001001895.1 |
| Ubash3b | NM_032873.3 |
| VCAM | NM_001078.3 |
| Xist | NR_001564.1 |
| Zap70 | NM_001079.3 |
| Zbtb16 | NM_006006.4 |
| Zbtb32 | NM_014383.1 |

Each of the steps of obtaining a tissue sample, preparing one or more tissue sections therefrom for a gene signature biomarker assay, performing the assay, and scoring the results may be performed by separate individuals/entities at separate locations. For example, a surgeon may obtain by biopsy a tissue sample from a cancer patient's tumor and then send the tissue sample to a pathology lab, which may fix the tissue sample and then prepare one or more slides, each with a single tissue section, for the assay. The slide(s) may be assayed soon after preparation, or stored for future assay. The lab that prepared a tissue section may conduct the assay or send the slide(s) to a different lab to conduct the assay. A pathologist or trained professional who scores the slide(s) for a PD-L1 gene signature may work for the diagnostic lab, or may be an independent contractor. Alternatively, a single diagnostic lab obtains the tissue sample from the subject's physician or surgeon and then performs all of the steps involved in preparing tissue sections, assaying the slide(s) and calculating the gene signature score for the tissue section(s).

In some embodiments, the individuals involved with preparing and assaying the tissue section for a gene signature biomarker do not know the identity of the subject whose sample is being tested; i.e., the sample received by the laboratory is made anonymous in some manner before being sent to the laboratory. For example, the sample may be merely identified by a number or some other code (a “sample ID”) and the results of the assay are reported to the party ordering the test using the sample ID. In preferred embodiments, the link between the identity of a subject and the subject's tissue sample is known only to the individual or to the individual's physician.

In some embodiments, after the test results have been obtained, the diagnostic laboratory generates a test report, which may comprise any one or both of the following results: the tissue sample was biomarker positive or negative, the gene signature score for the tumor sample and the reference score for that gene signature. The test report may also include a list of genes whose expression was analyzed in the assay.

In other embodiments, the test report may also include guidance on how to interpret the results for predicting if a subject is likely to respond to a PD-1 antagonist. For example, in one embodiment, the tested tumor sample is from a melanoma and has a PD-L1 gene signature score at or above a prespecified threshold, the test report may indicate that the subject has a score that is associated with response or better response to treatment with a PD-1 antagonist, while if the PD-L1 gene signature score is below the threshold, then the test report indicates that the patient has a score that is associated with no response or poor response to treatment with a PD-1 antagonist. In some embodiments, the prespecified threshold in melanoma tissue samples for the PD-L1 gene signature of Table 1 is equal to or greater than 1.87, 1.96 or 2.12.

In some embodiments, the test report is a written document prepared by the diagnostic laboratory and sent to the patient or the patient's physician as a hard copy or via electronic mail. In other embodiments, the test report is generated by a computer program and displayed on a video monitor in the physician's office. The test report may also comprise an oral transmission of the test results directly to the patient or the patient's physician or an authorized employee in the physician's office. Similarly, the test report may comprise a record of the test results that the physician makes in the patient's file.

Detecting the presence or absence of a PD-L1 gene signature of the invention may be performed using a kit that has been specially designed for this purpose. In one embodiment, the kit comprises a set of oligonucleotide probes capable of hybridizing to the target transcripts in the gene signature. The kit may further comprise oligonucleotide probes capable of detecting transcripts of other genes, such as control genes, or genes used for normalization purposes. The set of oligonucleotide probes may comprise an ordered array of oligonucleotides on a solid surface, such as a microchip, silica beads (such as BeadArray technology from Illumina, San Diego, Calif.), or a glass slide (see, e.g., WO 98/20020 and WO 98/20019). In some embodiments, the oligonucleotide probes are provided in one or more compositions in liquid or dried form.

Oligonucleotides in kits of the invention must be capable of specifically hybridizing to a target region of a polynucleotide, such as for example, an RNA transcript or cDNA generated therefrom. As used herein, specific hybridization means the oligonucleotide forms an anti-parallel double-stranded structure with the target region under certain hybridizing conditions, while failing to form such a structure with non-target regions when incubated with the polynucleotide under the same hybridizing conditions. The composition and length of each oligonucleotide in the kit will depend on the nature of the transcript containing the target region as well as the type of assay to be performed with the oligonucleotide and is readily determined by the skilled artisan.

In some embodiments, each oligonucleotide in the kit is a perfect complement of its target region. An oligonucleotide is said to be a “perfect” or “complete” complement of another nucleic acid molecule if every nucleotide of one of the molecules is complementary to the nucleotide at the corresponding position of the other molecule. While perfectly complementary oligonucleotides are preferred for detecting transcripts in a gene signature, departures from complete complementarity are contemplated where such departures do not prevent the molecule from specifically hybridizing to the target region as defined above. For example, an oligonucleotide probe may have one or more non-complementary nucleotides at its 5′ end or 3′ end, with the remainder of the probe being completely complementary to the target region. Alternatively, non-complementary nucleotides may be interspersed into the probe as long as the resulting probe is still capable of specifically hybridizing to the target region.

In some preferred embodiments, each oligonucleotide in the kit specifically hybridizes to its target region under stringent hybridization conditions. Stringent hybridization conditions are sequence-dependent and vary depending on the circumstances. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. As the target sequences are generally present in excess, at Tm, 50% of the probes are occupied at equilibrium.

Typically, stringent conditions include a salt concentration of at least about 0.01 to 1.0 M sodium ion concentration (or other salts) at pH 7.0 to 8.3 and the temperature is at least about 25° C. for short oligonucleotide probes (e.g., 10 to 50 nucleotides). Stringent conditions can also be achieved with the addition of destabilizing agents such as formamide. For example, conditions of 5×SSPE (750 mM NaCl, 50 mM NaPhosphate, 5 mM EDTA, pH 7.4) and a temperature of 25-30° C. are suitable for allele-specific probe hybridizations. Additional stringent conditions can be found in Molecular Cloning: A Laboratory Manual, Sambrook et al., Cold Spring Harbor Press, Cold Spring Harbor, N.Y. (1989), chapters 7, 9, and 11, and in NUCLEIC ACID HYBRIDIZATION, A PRACTICAL APPROACH, Haymes et al., IRL Press, Washington, D.C., 1985.

One non-limiting example of stringent hybridization conditions includes hybridization in 4× sodium chloride/sodium citrate (SSC), at about 65-70° C. (or alternatively hybridization in 4×SSC plus 50% formamide at about 42-50° C.) followed by one or more washes in 1×SSC, at about 65-70° C. A non-limiting example of highly stringent hybridization conditions includes hybridization in 1×SSC, at about 65-70° C. (or alternatively hybridization in 1×SSC plus 50% formamide at about 42-50° C.) followed by one or more washes in 0.3×SSC, at about 65-70° C. A non-limiting example of reduced stringency hybridization conditions includes hybridization in 4×SSC, at about 50-60° C. (or alternatively hybridization in 6×SSC plus 50% formamide at about 40-45° C.) followed by one or more washes in 2×SSC, at about 50-60° C. Stringency conditions with ranges intermediate to the above-recited values, e.g., at 65-70° C. or at 42-50° C. are also intended to be encompassed by the present invention. SSPE (1×SSPE is 0.15M NaCl, 10 mM NaH₂PO₄, and 1.25 mM EDTA, pH 7.4) can be substituted for SSC (1×SSC is 0.15M NaCl and 15 mM sodium citrate) in the hybridization and wash buffers; washes are performed for 15 minutes each after hybridization is complete.

The hybridization temperature for hybrids anticipated to be less than 50 base pairs in length should be 5-10° C. less than the melting temperature (Tₘ) of the hybrid, where Tm is determined according to the following equations. For hybrids less than 18 base pairs in length, Tₘ (° C.)=2(# of A+T bases)+4(# of G+C bases). For hybrids between 18 and 49 base pairs in length, Tₘ (° C.)=81.5+16.6 (log₁₀[Na+])+0.41 (% G+C)−(600/N), where N is the number of bases in the hybrid, and [Na+] is the concentration of sodium ions in the hybridization buffer ([Na+] for 1×SSC=0.165 M).

The oligonucleotides in kits of the invention may be comprised of any phosphorylation state of ribonucleotides, deoxyribonucleotides, and acyclic nucleotide derivatives, and other functionally equivalent derivatives. Alternatively, the oligonucleotides may have a phosphate-free backbone, which may be comprised of linkages such as carboxymethyl, acetamidate, carbamate, polyamide (peptide nucleic acid (PNA)) and the like (Varma, in MOLECULAR BIOLOGY AND BIOTEChNOLOGY, A COMPREHENSIVE DESK REFERENCE, Meyers, ed., pp. 6 17-20, VCH Publishers, Inc., 1995). The oligonucleotides may be prepared by chemical synthesis using any suitable methodology known in the art, or may be derived from a biological sample, for example, by restriction digestion. The oligonucleotides may contain a detectable label, according to any technique known in the art, including use of radiolabels, fluorescent labels, enzymatic labels, proteins, haptens, antibodies, sequence tags and the like. The oligonucleotides in the kit may be manufactured and marketed as analyte specific reagents (ASRs) or may be constitute components of an approved diagnostic device.

Kits of the invention may also contain other reagents such as hybridization buffer and reagents to detect when hybridization with a specific target molecule has occurred. Detection reagents may include biotin- or fluorescent-tagged oligonucleotides and/or an enzyme-labeled antibody and one or more substrates that generate a detectable signal when acted on by the enzyme. It will be understood by the skilled artisan that the set of oligonucleotides and reagents for performing the assay will be provided in separate receptacles placed in the kit container if appropriate to preserve biological or chemical activity and enable proper use in the assay.

In other embodiments, each of the oligonucleotide probes and all other reagents in the kit have been quality tested for optimal performance in an assay designed to determine the PD-L1 gene signature score in a tumor sample, and preferably when the tumor sample is an FFPE tissue section. In some embodiments, the kit includes an instruction manual that describes how to use the determined gene signature score to assign, to the tested tumor sample, the presence or absence of a gene signature biomarker that predicts response to treatment with a PD-1 antagonist.

### B. Pharmaceutical Compositions, Drug Products and Treatment Regimens

An individual to be treated by any of the methods and products described herein is a human subject diagnosed with a tumor, and a sample of the subject's tumor is available or obtainable to use in testing for the presence or absence of any of the gene signature biomarkers described herein.

The tumor tissue sample can be collected from a subject before and/or after exposure of the subject to one or more therapeutic treatment regimens, such as for example, a PD-1 antagonist, a chemotherapeutic agent, radiation therapy. Accordingly, tumor samples may be collected from a subject over a period of time. The tumor sample can be obtained by a variety of procedures including, but not limited to, surgical excision, aspiration or biopsy.

A physician may use a PD-L1 gene signature score as a guide in deciding how to treat a patient who has been diagnosed with a type of cancer that is susceptible to treatment with a PD-1 antagonist or other chemotherapeutic agent(s). Prior to initiation of treatment with the PD-1 antagonist or the other chemotherapeutic agent(s), the physician would typically order a diagnostic test to determine if a tumor tissue sample removed from the patient is positive or negative for a PD-L1 gene signature biomarker. However, it is envisioned that the physician could order a first or subsequent diagnostic tests at any time after the individual is administered the first dose of the PD-1 antagonist or other chemotherapeutic agent(s). In some embodiments, a physician may be considering whether to treat the patient with a pharmaceutical product that is indicated for patients whose tumor tests positive for the gene signature biomarker. For example, if the reported score is at or above a pre-specified threshold score that is associated with response or better response to treatment with a PD-1 antagonist, the patient is treated with a therapeutic regimen that includes at least the PD-1 antagonist (optionally in combination with one or more chemotherapeutic agents), and if the reported gene signature score is below a pre-specified threshold score that is associated with no response or poor response to treatment with a PD-1 antagonist, the patient is treated with a therapeutic regimen that does not include any PD-1 antagonist.

In deciding how to use the PD-L1 gene signature test results in treating any individual patient, the physician may also take into account other relevant circumstances, such as the stage of the cancer, weight, gender, and general condition of the patient, including inputting a combination of these factors and the gene signature biomarker test results into a model that helps guide the physician in choosing a therapy and/or treatment regimen with that therapy.

The physician may choose to treat the patient who tests biomarker positive with a combination therapy regimen that includes a PD-1 antagonist and one or more additional therapeutic agents. The additional therapeutic agent may be, e.g., a chemotherapeutic, a biotherapeutic agent (including but not limited to antibodies to VEGF, EGFR, Her2/neu, VEGF receptors, other growth factor receptors, CD20, CD40, CD-40L, CTLA-4, OX-40, 4-1BB, and ICOS), an immunogenic agent (for example, attenuated cancerous cells, tumor antigens, antigen presenting cells such as dendritic cells pulsed with tumor derived antigen or nucleic acids, immune stimulating cytokines (for example, IL-2, IFNα2, GM-CSF), and cells transfected with genes encoding immune stimulating cytokines such as but not limited to GM-CSF).

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBI-TMI); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as the enediyne antibiotics (e.g. calicheamicin, especially calicheamicin gamma1I and calicheamicin phiI1, see, e.g., Agnew, Chem. Intl. Ed. Engl., 33:183-186 (1994); dynemicin, including dynemicin A; bisphosphonates, such as clodronate; an esperamicin; as well as neocarzinostatin chromophore and related chromoprotein enediyne antibiotic chromomophores), aclacinomysins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidamine; maytansinoids such as maytansine and ansamitocins; mitoguazone; mitoxantrone; mopidamol; nitracrine; pentostatin; phenamet; pirarubicin; losoxantrone; podophyllinic acid; 2-ethylhydrazide; procarbazine; razoxane; rhizoxin; sizofuran; spirogermanium; tenuazonic acid; triaziquone; 2, 2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g. paclitaxel and doxetaxel; chlorambucil; gemcitabine; 6-thioguanine; mercaptopurine; methotrexate; platinum analogs such as cisplatin and carboplatin; vinblastine; platinum; etoposide (VP-16); Ifosfamide; mitoxantrone; vincristine; vinorelbine; novantrone; teniposide; edatrexate; daunomycin; aminopterin; xeloda; ibandronate; CPT-11; topoisomerase inhibitor RFS 2000; difluoromethylornithine (DMFO); retinoids such as retinoic acid; capecitabine; and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen, raloxifene, droloxifene, 4-hydroxytamoxifen, trioxifene, keoxifene, LY117018, onapristone, and toremifene (Fareston); aromatase inhibitors that inhibit the enzyme aromatase, which regulates estrogen production in the adrenal glands, such as, for example, 4(5)-imidazoles, aminoglutethimide, megestrol acetate, exemestane, formestane, fadrozole, vorozole, letrozole, and anastrozole; and anti-androgens such as flutamide, nilutamide, bicalutamide, leuprolide, and goserelin; and pharmaceutically acceptable salts, acids or derivatives of any of the above.

Each therapeutic agent in a combination therapy used to treat a biomarker positive patient may be administered either alone or in a medicament (also referred to herein as a pharmaceutical composition) which comprises the therapeutic agent and one or more pharmaceutically acceptable carriers, excipients and diluents, according to standard pharmaceutical practice.

Each therapeutic agent in a combination therapy used to treat a biomarker positive patient may be administered simultaneously (i.e., in the same medicament), concurrently (i.e., in separate medicaments administered one right after the other in any order) or sequentially in any order. Sequential administration is particularly useful when the therapeutic agents in the combination therapy are in different dosage forms (one agent is a tablet or capsule and another agent is a sterile liquid) and/or are administered on different dosing schedules, e.g., a chemotherapeutic that is administered at least daily and a biotherapeutic that is administered less frequently, such as once weekly, once every two weeks, or once every three weeks.

In some embodiments, at least one of the therapeutic agents in the combination therapy is administered using the same dosage regimen (dose, frequency and duration of treatment) that is typically employed when the agent is used as monotherapy for treating the same cancer. In other embodiments, the patient receives a lower total amount of at least one of the therapeutic agents in the combination therapy than when the agent is used as monotherapy, e.g., smaller doses, less frequent doses, and/or shorter treatment duration.

Each therapeutic agent in a combination therapy used to treat a biomarker positive patient can be administered orally or parenterally, including the intravenous, intramuscular, intraperitoneal, subcutaneous, rectal, topical, and transdermal routes of administration.

A patient may be administered a PD-1 antagonist prior to or following surgery to remove a tumor and may be used prior to, during or after radiation therapy.

In some embodiments, a PD-1 antagonist is administered to a patient who has not been previously treated with a biotherapeutic or chemotherapeutic agent, i.e., is treatment-naïve. In other embodiments, the PD-1 antagonist is administered to a patient who failed to achieve a sustained response after prior therapy with a biotherapeutic or chemotherapeutic agent, i.e., is treatment-experienced.

A therapy comprising a PD-1 antagonist is typically used to treat a tumor that is large enough to be found by palpation or by imaging techniques well known in the art, such as MRI, ultrasound, or CAT scan. In some preferred embodiments, the therapy is used to treat an advanced stage tumor having dimensions of at least about 200 mm³, 300 mm³, 400 mm³, 500 mm³, 750 mm³, or up to 1000 mm³.

Selecting a dosage regimen (also referred to herein as an administration regimen) for a therapy comprising a PD-1 antagonist depends on several factors, including the serum or tissue turnover rate of the entity, the level of symptoms, the immunogenicity of the entity, and the accessibility of the target cells, tissue or organ in the individual being treated. Preferably, a dosage regimen maximizes the amount of the PD-1 antagonist that is delivered to the patient consistent with an acceptable level of side effects. Accordingly, the dose amount and dosing frequency depends in part on the particular PD-1 antagonist, any other therapeutic agents to be used, and the severity of the cancer being treated, and patient characteristics. Guidance in selecting appropriate doses of antibodies, cytokines, and small molecules are available. See, e.g., Wawrzynczak (1996) Antibody Therapy, Bios Scientific Pub. Ltd, Oxfordshire, UK; Kresina (ed.) (1991) Monoclonal Antibodies, Cytokines and Arthritis, Marcel Dekker, New York, N.Y.; Bach (ed.) (1993) Monoclonal Antibodies and Peptide Therapy in Autoimmune Diseases, Marcel Dekker, New York, N.Y.; Baert et al. (2003) New Engl. J. Med. 348:601-608; Milgrom et al. (1999) New Engl. J. Med. 341:1966-1973; Slamon et al. (2001) New Engl. J. Med. 344:783-792; Beniaminovitz et al. (2000) New Engl. J. Med. 342:613-619; Ghosh et al. (2003) New Engl. J. Med. 348:24-32; Lipsky et al. (2000) New Engl. J. Med. 343:1594-1602; Physicians' Desk Reference 2003 (Physicians' Desk Reference, 57th Ed); Medical Economics Company; ISBN: 1563634457; 57th edition (November 2002). Determination of the appropriate dosage regimen may be made by the clinician, e.g., using parameters or factors known or suspected in the art to affect treatment or predicted to affect treatment, and will depend, for example, the patient's clinical history (e.g., previous therapy), the type and stage of the cancer to be treated and biomarkers of response to one or more of the therapeutic agents in the combination therapy.

Biotherapeutic agents used in combination with a PD-1 antagonist may be administered by continuous infusion, or by doses at intervals of, e.g., daily, every other day, three times per week, or one time each week, two weeks, three weeks, monthly, bimonthly, etc. A total weekly dose is generally at least 0.05 μg/kg, 0.2 μg/kg, 0.5 μg/kg, 1 μg/kg, 10 μg/kg, 100 μg/kg, 0.2 mg/kg, 1.0 mg/kg, 2.0 mg/kg, 10 mg/kg, 25 mg/kg, 50 mg/kg body weight or more. See, e.g., Yang et al. (2003) New Engl. J. Med. 349:427-434; Herold et al. (2002) New Engl. J. Med. 346:1692-1698; Liu et al. (1999) J. Neurol. Neurosurg. Psych. 67:451-456; Portielji et al. (20003) Cancer Immunol. Immunother. 52:133-144.

In some embodiments that employ an anti-human PD-1 mAb as the PD-1 antagonist, the dosing regimen will comprise administering the anti-human PD-1 mAb at a dose of 1, 2, 3, 5 or 10 mg/kg at intervals of about 14 days (±2 days) or about 21 days (±2 days) or about 30 days (±2 days) throughout the course of treatment.

In other embodiments that employ an anti-human PD-1 mAb as the PD-1 antagonist, the dosing regimen will comprise administering the anti-human PD-1 mAb at a dose of from about 0.005 mg/kg to about 10 mg/kg, with intra-patient dose escalation. In other escalating dose embodiments, the interval between doses will be progressively shortened, e.g., about 30 days (±2 days) between the first and second dose, about 14 days (±2 days) between the second and third doses. In certain embodiments, the dosing interval will be about 14 days (±2 days), for doses subsequent to the second dose.

In certain embodiments, a subject will be administered an intravenous (IV) infusion of a medicament comprising any of the PD-1 antagonists described herein, and such administration may be part of a treatment regimen employing the PD-1 antagonist as a monotherapy regimen or as part of a combination therapy.

In one preferred embodiment of the invention, the PD-1 antagonist is nivolumab, which is administered intravenously at a dose selected from the group consisting of: 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W.

In another preferred embodiment of the invention, the PD-1 antagonist is MK-3475, which is administered in a liquid medicament at a dose selected from the group consisting of 1 mg/kg Q2W, 2 mg/kg Q2W, 3 mg/kg Q2W, 5 mg/kg Q2W, 10 mg Q2W, 1 mg/kg Q3W, 2 mg/kg Q3W, 3 mg/kg Q3W, 5 mg/kg Q3W, and 10 mg Q3W. In some particularly preferred embodiments, MK-3475 is administered as a liquid medicament which comprises 25 mg/ml MK-3475, 7% (w/v) sucrose, 0.02% (w/v) polysorbate 80 in 10 mM histidine buffer pH 5.5, and the selected dose of the medicament is administered by IV infusion over a time period of 30 minutes. The optimal dose for MK-3475 in combination with any other therapeutic agent may be identified by dose escalation starting with 2 mg/kg and going up to 10 mg/kg.

The present invention also provides a medicament which comprises a PD-1 antagonist as described above and a pharmaceutically acceptable excipient. When the PD-1 antagonist is a biotherapeutic agent, e.g., a mAb, the antagonist may be produced in CHO cells using conventional cell culture and recovery/purification technologies.

In some embodiments, a medicament comprising an anti-PD-1 antibody as the PD-1 antagonist may be provided as a liquid formulation or prepared by reconstituting a lyophilized powder with sterile water for injection prior to use. WO 2012/135408 describes the preparation of liquid and lyophilized medicaments comprising MK-3475 that are suitable for use in the present invention. In some preferred embodiments, a medicament comprising MK-3475 is provided in a glass vial which contains about 50 mg of MK-3475.

### Exemplary Specific Embodiments of the Invention

1. A method for testing a tumor for the presence or absence of a biomarker that predicts response to treatment with a PD-1 antagonist, which comprises:

obtaining a sample from the tumor, measuring the raw RNA expression level in the tumor sample for each gene in a PD-L1 gene signature;

normalizing each of the measured raw RNA expression levels; and

calculating the arithmetic mean of the normalized RNA expression levels for each of the genes to generate a score for the PD-L1 gene signature;

wherein the PD-L1 gene signature comprises PD-L1 and at least five other genes in Table 1.
2. The method of embodiment 1, wherein the method further comprises:

comparing the calculated score to a reference score for the PD-L1 gene signature; and

classifying the tumor as biomarker positive or biomarker negative;

wherein if the calculated score is equal to or greater than the reference score, then the tumor is classified as biomarker positive, and if the calculated PD-L1 gene signature score is less than the reference PD-L1 gene signature score, then the tumor is classified as biomarker negative.
3. A method for treating a subject having a tumor which comprises:

determining if the tumor is positive or negative for a PD-L1 gene signature biomarker; and

administering to the subject a PD-1 antagonist if the tumor is positive for the biomarker; or

administering to the subject a cancer treatment that does not include a PD-1 antagonist if the tumor is negative for the biomarker;

wherein the PD-L1 gene signature comprises PD-L1 and at least five other genes in Table 1.
4. The method of embodiment 3, wherein the determining step comprises:

obtaining a sample from the subject's tumor;

sending the tumor sample to a laboratory with a request to test the sample for the presence or absence of a PD-L1 gene signature biomarker; and

receiving a report from the laboratory that states whether the tumor sample is biomarker positive or biomarker negative.

5. A method for treating a subject having a tumor which comprises:

obtaining a sample from the tumor;

measuring the raw RNA expression level in the tumor sample for each gene in a PD-L1 gene signature;

normalizing each of the measured raw RNA expression levels;

calculating the arithmetic mean of the normalized RNA expression levels for each of the genes to generate a score for the PD-L1 gene signature; and

administering to the subject a PD-1 antagonist if the calculated score is equal to or greater than a reference score for the PD-L1 gene signature; or

administering to the subject a cancer therapy that does not include a PD-1 antagonist if the calculated score is less than the reference score;

wherein the PD-L1 gene signature comprises PD-L1 and at least five other genes in Table 1.
6. A pharmaceutical composition comprising a PD-1 antagonist for use in a subject who has a tumor that tests positive for a PD-L1 gene signature biomarker, wherein the PD-L1 gene signature comprises PD-L1 and at least five other genes in Table 1.
7. A drug product which comprises a pharmaceutical composition and prescribing information, wherein the pharmaceutical composition comprises a PD-1 antagonist and at least one pharmaceutically acceptable excipient and the prescribing information states that the pharmaceutical composition is indicated for use in a subject who has a tumor that tests positive for a PD-L1 gene signature biomarker.
8. The pharmaceutical composition of embodiment 6 or the drug product of embodiment 7, wherein the positive biomarker test result was generated by a method comprising:

obtaining a sample from the tumor,

measuring the raw RNA expression level in the tumor sample for each gene in a PD-L1 gene signature;

normalizing each of the measured raw RNA expression levels;

calculating the arithmetic mean of the normalized RNA expression levels for each of the genes to generate a score for the PD-L1 gene signature;

comparing the calculated score to a reference score for the PD-L1 gene signature; and

classifying the tumor as biomarker positive or biomarker negative;

wherein if the calculated score is equal to or greater than the reference score, then the tumor is classified as biomarker positive, and if the calculated PD-L1 gene signature score is less than the reference PD-L1 gene signature score, then the tumor is classified as biomarker negative.
9. A kit for assaying a tumor sample to determine a PD-L1 gene signature score for the tumor sample, wherein the kit comprises a first set of probes for detecting expression of each gene in the PD-L1 gene signature, wherein the PD-L1 gene signature comprises PD-L1 and at least five other genes in Table 1.
10. The kit of embodiment 9, wherein the first set of probes is designed to detect expression of the transcripts listed in Table 1 for PD-L1, PD-L2, STAT1, LAG3, CXCL10, and CLEC10a.
11. The kit of embodiments 9 or 10, which further comprises a second set of probes for detecting target transcripts expressed in the tumor sample by a set of normalization genes.
12. The method, composition, drug product or kit of any of the above embodiments, wherein the measuring step comprises contacting RNA molecules in the sample with at least one probe for the transcript listed in Table 1 for each gene whose expression is to be measured, wherein the contacting is performed under stringent hybridization conditions, and quantitating the number of probe-RNA hybrids generated in the contacting step.
13. The method, composition, drug product or kit of any of the above embodiments, wherein the measuring step comprises amplifying and quantifying the transcript listed in Table 1 for each gene whose expression is to be measured.
14. The method, composition, drug product or kit of any of the above embodiments, wherein the normalizing step comprises performing quantile normalization of raw RNA expression values relative to the distribution of raw RNA expression values in the test sample and a plurality of control samples for a set of normalization genes, followed by a subsequent log 10-transformation.
15. The method, composition, drug product or kit of any of the above embodiments, wherein the normalization gene set consists essentially of at least 100 or 200 genes in the 400 gene set listed in Table 4.
16. The method, composition, drug product or kit of any of the above embodiments, wherein the set of normalization genes consists essentially of at least 300 or 400 genes in the 400 gene set listed in Table 4.
17. The method, composition, drug product or kit of any of the above embodiments, wherein the PD-L1 gene signature consists essentially of PD-L1, PD-L2, STAT1, LAG3, CXCL10, and CLEC10a.
18. The method, composition, drug product or kit of any of the above embodiments, wherein the reference score is pre-selected to divide the majority of responders to the PD-1 antagonist from the majority of non-responders to the PD-1 antagonist.
19. The method, composition, drug product or kit of any of the above embodiments, wherein the majority of responders achieved at least a partial response to the PD-1 antagonist as measured by RECIST 1.1.
20. The method, composition, drug product or kit of any of the above embodiments, wherein the majority of responders achieved a complete response to the PD-1 antagonist as measured by RECIST 1.1.
21. The method, composition, drug product or kit of any of the above embodiments, wherein the PD-L1 gene signature consists essentially of PD-L1, PD-L2, STAT1, LAG3, CXCL10, and CLEC10a, the test and reference PD-L1 gene signature scores are determined by performing quantile normalization of raw RNA expression values relative to the distribution of raw RNA expression values for a set of at least 300 normalization genes in the test tumor sample and in a plurality of control tumor samples followed by a subsequent log 10-transformation.
22. The method, composition, drug product or kit of embodiment 21, wherein the tumor is metastatic melanoma, the set of normalization genes consists essentially of the 400 genes in Table 4 and the reference score is between about 1.87 and about 2.12, between about 1.96 and about 2.12, or is about 2.12.
23. The method, composition, drug product or kit of any of the above embodiments, wherein the PD-1 antagonist is a monoclonal antibody, or an antigen binding fragment thereof, which specifically binds to PD-1 or to PD-L1 and blocks the binding of PD-L1 to PD-1.
24. The method, composition, drug product or kit of embodiment 23, wherein the PD-1 antagonist is an anti-PD-1 monoclonal antibody which comprises a heavy chain and a light chain, wherein the heavy and light chains comprise SEQ ID NO:21 and SEQ ID NO:22.
25. The method, composition, drug product or kit of embodiment 22, wherein the PD-1 antagonist is MK-3475 and the reference score is about 2.1.
26. The method, composition, drug product or kit of embodiment 22, wherein the PD-1 antagonist is MPDL3280A, BMS-936559, MEDI4736, MSB0010718C or a monoclonal antibody which comprises the heavy chain and light chain variable regions of SEQ ID NO:24 and SEQ ID NO:21, respectively, of WO2013/019906.
27. The method, composition, drug product or kit of embodiment 22, wherein the monoclonal antibody, or antigen binding fragment thereof, comprises: (a) light chain CDRs of SEQ ID NOs: 1, 2 and 3 and heavy chain CDRs of SEQ ID NOs: 4, 5 and 6; or (b) light chain CDRs of SEQ ID NOs: 7, 8 and 9 and heavy chain CDRs of SEQ ID NOs: 10, 11 and 12.
28. The method, composition, drug product or kit of embodiment 22, wherein the PD-1 antagonist is an anti-PD-1 monoclonal antibody which comprises a heavy chain and a light chain, and wherein the heavy chain comprises SEQ ID NO:23 and the light chain comprises SEQ ID NO:24.
29. The method, composition, drug product or kit of any of the above embodiments, wherein the tumor sample is from a subject with ipilimumab-naïve advanced melanoma or ipilimumab-refractory advanced melanoma.
30. The method, composition, drug product or kit of any of the above embodiments, wherein the PD-1 antagonist is MK-3475 or nivolumab.
31. The method, composition, drug product or kit of any of the above embodiments, wherein the reference score is selected to provide a negative predictive value that is greater than the positive predictive value.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, N.Y.; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, N.Y., pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, N.Y.; Kay et al. (1996) Phage Display of Peptides and Proteins: A Laboratory Manual, Academic Press, San Diego, Calif.; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, Oreg.; Sigma-Aldrich (2003) Catalogue, St. Louis, Mo.).

Standard methods of histology of the immune system are described (see, e.g., Muller-Harmelink (ed.) (1986) Human Thymus: Histopathology and Pathology, Springer Verlag, New York, N.Y.; Hiatt, et al. (2000) Color Atlas of Histology, Lippincott, Williams, and Wilkins, Phila, Pa.; Louis, et al. (2002) Basic Histology: Text and Atlas, McGraw-Hill, New York, N.Y.).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, Md.); GCG Wisconsin Package (Accelrys, Inc., San Diego, Calif.); DeCypher® (TimeLogic Corp., Crystal Bay, Nev.); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### EXAMPLES

### Example 1

### Preparation of FFPE Whole Cell Lysates and Subsequent Gene Expression Analysis Using the NanoString nCounter™ System

This example describes the methods used to analyze gene expression in the FFPE tumor samples discussed in the Examples below. Whole cell lysates were prepared from slides of FFPE tissue for analysis on the NanoString nCounter™ gene expression platform (NanoString Technologies, Seattle, Wash.). Prior to making the cell lysate, each tissue section was deparaffinized in xylene for 3×5 min and then rehydrated by immersing consecutively in 100% ethanol for 2×2 min, 95% ethanol for 2 min, 70% ethanol for 2 min and then immersed in dH₂O until ready to be processed. Tissue was lysed on the slide by adding 10-50 ul of PKD buffer (Qiagen catalog #73504). Tissue was scraped from the slide and transferred to a 1.5 ml eppendorf tube. Proteinase K (Qiagen catalog #73504) was added at no more than 10% final volume and the RNA lysate was incubated for 15 min at 55° C. and then 15 min at 80° C. The RNA lysate was stored at −80° C. until gene expression profiling was performed using the NanoString nCounter™ system.

For each tumor sample, 5 ul of cellular lysate was mixed with a set of 400 capture and reporter probe pairs designed by NanoString for a set of 400 genes specified by the inventors herein. Each capture probe was biotinylated on its 3′ end and the 5′ end of each reporter probe was tagged with a fluorescent barcode. Probes and lysate were hybridized overnight at 65° C. for 12-16 hours as per NanoString's recommendations. Hybridized samples were run on the NanoString nCounter™ preparation station using NanoString's high sensitivity protocol, in which excess capture and reporter probes are removed and transcript-specific ternary complexes are immobilized on a streptavidin-coated cartridge. The samples were scanned at maximum scan resolution capabilities using the nCounter™ Digital Analyzer.

### Example 2

### Discovery of a PD-L1 Co-Expressed Gene Signature

The inventors herein selected the 400 gene set listed in Table 4 to investigate whether a gene expression signature could be derived for genes that are co-expressed with PD-L1 in multiple tumor types and that would be useful in predicting which patients are more likely to have an anti-tumor response to therapy with a PD-1 antagonist.

Tumor samples that had been obtained from the patients prior to treatment with MK-3475 were assayed for expression of the 400 gene set in Table 4 using the NanoString nCounter® Analysis System and a CodeSet designed by NanoString and a CodeSet designed by NanoString to measure expression of the gene set in a single multiplex reaction for each FFPE tumor sample. The CodeSet included the target transcript listed in Table 4 and a pair of capture and reporter probes for that transcript for each of the 400 genes. For each patient tumor sample, the raw transcript expression counts data were normalized by performing quantile normalization relative to the reference distribution and subsequent log 10-transformation. The reference distribution was generated by pooling reported counts for all samples after excluding values for technical (both positive and negative control) probes, and without performing intermediate normalization relying on negative (background-adjusted) or positive (synthetic sequences spiked with known titrations).

The normalized expression results for all 400 genes were reviewed and five genes were selected to include in a PD-L1 gene signature based on a strong positive Pearson correlation of expression of each of these genes with PD-L1 expression in melanoma (MEL), large cell lung cancer (LCLC) and bladder cancer as shown in Table 5 below. The number of patient tumors for which expression data were evaluated is shown in parenthesis.

TABLE 5
Characteristics of a preferred PD-L1 Gene Signature of the
Invention

| Gene | | Pearson Correlation to PD-L1 | | | |
|---|---|---|---|---|---|
| | Target | | | | Bladder |
| Symbol | Transcript | MEL (75) | MEL (20) | LCLC (27) | (30) |
| PDL-1 | NM_014143 | 100% | 100% | 100% | 100% |
| PDL-2 | NM_025239 | 71% | 72% | 70% | 70% |
| LAG3 | NM_002286 | 61% | 36% | 33% | 40% |
| STAT1 | NM_007315 | 57% | 26% | 30% | 50% |
| CXCL10 | NM_001565 | 55% | 23% | 34% | 56% |
| CLEC10a | NM_182906 | 11% | 61% | 63% | 18% |

### Example 3

### Discovery of a PD-L1 Gene Signature Score that Predicts Response to MK-3475 Response

The ability of the PD-L1 gene signature discovered in Example 2 to predict response to a PD-1 antagonist was evaluated using clinical response data for a cohort of 19 melanoma patients who had been treated with MK-3475.

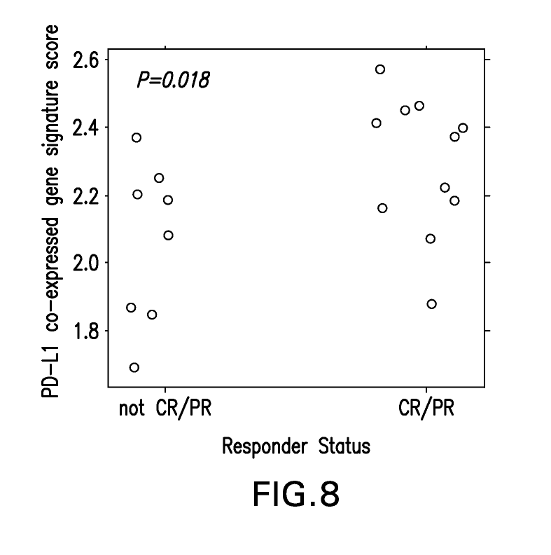

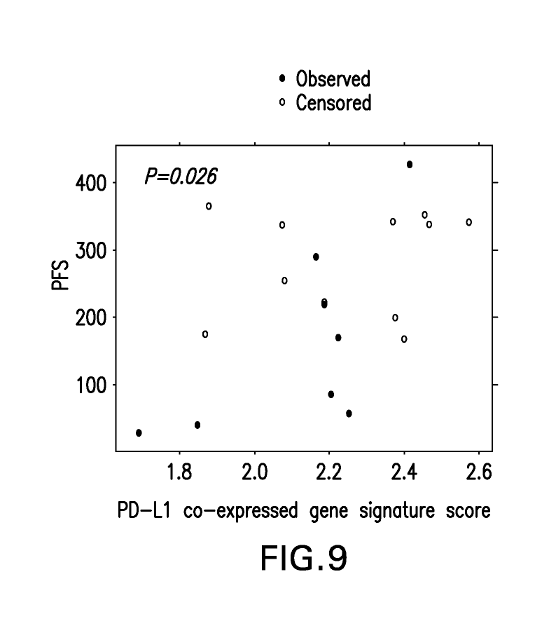

This 19 patient cohort was divided into a group of 11 Responders (patients whose best overall response (OR) was a complete response (CR) or partial response (PR) to MK-3475, each as determined by an independent reviewer using RECIST 1.1 criteria) and a group of 8 Non-responders (whose best OR was not a CR or PR). Tumor samples that had been obtained from the patients prior to treatment with MK-3475 were assayed for expression of the 400 gene set in Table 4 using the NanoString nCounter® Analysis System. A PD-L1 gene signature score for each patient tumor sample was calculated as the arithmetic mean of the quantile normalized gene expression amount for each of the six transcripts listed in Table 5. Association between PD-L1 gene signature score and best overall response to MK-3475 treatment was assessed using a one-sided t-test analysis for Response vs. Non-response (FIG. 8) and a cox-regression analysis for length of progression free survival (PFS) (FIG. 9). The results of these analyses demonstrated statistically significant associations between higher PD-L1 gene signature scores and better clinical responses to MK-3475.

The inventors herein evaluated the potential utility of this PD-L1 gene signature in selecting patients for therapy with a PD-1 antagonist by comparing the PD-L1 gene signature scores for samples from the 19 patient cohort with scores for the same PD-L1 gene signature determined for an independent set of melanoma tumors. The range of PD-L1 gene signature scores determined for these two tumor groups are shown in Table 6, with the shaded rows indicating a set of scores that may be useful as a cut-off point, or reference gene signature score, to classify between about 30% and 60% of melanoma tumor samples as biomarker positive, and thus more likely to respond to treatment with MK-3475.

TABLE 6
Range of PD-L1 Gene Signature Scores in 2 Different
Melanoma Patient Sets

| PD-L1 Co-Expressed | | |
|---|---|---|
| Gene Signature | Melanoma-19 | Melanoma-71 Independent |
| 1.3704 | 0% | 0% |
| 1.4427 | 0% | 0% |
| 1.4777 | 0% | 1% |
| 1.5147 | 0% | 1% |
| 1.5377 | 0% | 1% |
| 1.5484 | 0% | 3% |
| 1.5641 | 0% | 4% |
| 1.5831 | 0% | 6% |
| 1.5968 | 0% | 6% |
| 1.6127 | 0% | 6% |
| 1.6236 | 5% | 6% |
| 1.6346 | 5% | 7% |
| 1.6438 | 5% | 10% |
| 1.6582 | 5% | 10% |
| 1.6675 | 5% | 10% |
| 1.675 | 5% | 10% |
| 1.6856 | 5% | 10% |
| 1.7099 | 5% | 13% |
| 1.7186 | 5% | 14% |
| 1.7269 | 5% | 15% |
| 1.7356 | 5% | 17% |
| 1.7459 | 5% | 18% |
| 1.7496 | 5% | 20% |
| 1.7679 | 5% | 21% |
| 1.7694 | 5% | 21% |
| 1.7809 | 5% | 23% |
| 1.7856 | 5% | 24% |
| 1.7911 | 10% | 24% |
| 1.8014 | 10% | 24% |
| 1.8105 | 15% | 24% |
| 1.8135 | 20% | 24% |
| 1.8223 | 20% | 25% |
| 1.8268 | 20% | 25% |
| 1.8392 | 20% | 27% |
| 1.8497 | 20% | 27% |
| 1.8577 | 20% | 28% |
| 1.8713 | 20% | 30% |
| 1.8756 | 20% | 30% |
| 1.883 | 20% | 30% |
| 1.8921 | 20% | 31% |
| 1.8987 | 20% | 32% |
| 1.9048 | 20% | 32% |
| 1.9136 | 20% | 32% |
| 1.9283 | 20% | 34% |
| 1.9322 | 20% | 37% |
| 1.9382 | 20% | 38% |
| 1.9517 | 20% | 38% |
| 1.9636 | 20% | 41% |
| 1.9675 | 20% | 41% |
| 1.979 | 20% | 42% |
| 1.9956 | 20% | 44% |
| 2.006 | 20% | 45% |
| 2.0151 | 20% | 48% |
| 2.0284 | 25% | 48% |
| 2.0438 | 30% | 48% |
| 2.0539 | 30% | 52% |
| 2.0614 | 30% | 55% |
| 2.0729 | 30% | 55% |
| 2.0812 | 30% | 58% |
| 2.0968 | 30% | 62% |
| 2.118 | 30% | 63% |
| 2.1278 | 35% | 65% |
| 2.1318 | 40% | 66% |
| 2.1479 | 40% | 68% |
| 2.1591 | 45% | 68% |
| 2.1697 | 50% | 69% |
| 2.1726 | 50% | 69% |
| 2.1828 | 50% | 69% |
| 2.1913 | 50% | 70% |
| 2.1989 | 60% | 70% |
| 2.2067 | 60% | 72% |
| 2.2124 | 60% | 73% |
| 2.2253 | 60% | 75% |
| 2.2286 | 60% | 76% |
| 2.2331 | 65% | 76% |
| 2.2468 | 65% | 76% |
| 2.2503 | 65% | 76% |
| 2.2571 | 65% | 77% |
| 2.2644 | 65% | 79% |
| 2.2735 | 65% | 80% |
| 2.2819 | 65% | 82% |
| 2.2902 | 65% | 83% |
| 2.2981 | 65% | 83% |
| 2.306 | 65% | 83% |
| 2.3134 | 65% | 83% |
| 2.3209 | 65% | 85% |
| 2.3286 | 70% | 85% |
| 2.3351 | 70% | 87% |
| 2.3442 | 70% | 87% |
| 2.3517 | 70% | 89% |
| 2.3593 | 75% | 90% |
| 2.3737 | 75% | 92% |
| 2.379 | 80% | 92% |
| 2.3869 | 85% | 92% |
| 2.4017 | 85% | 93% |
| 2.4172 | 85% | 93% |
| 2.4343 | 85% | 94% |
| 2.4382 | 95% | 94% |
| 2.4944 | 95% | 96% |
| 2.5897 | 100% | 97% |
| 2.7962 | 100% | 100% |

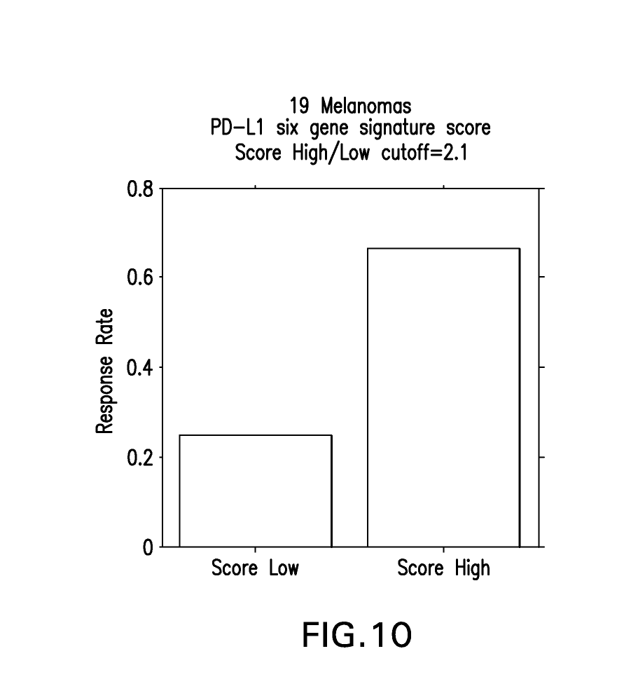

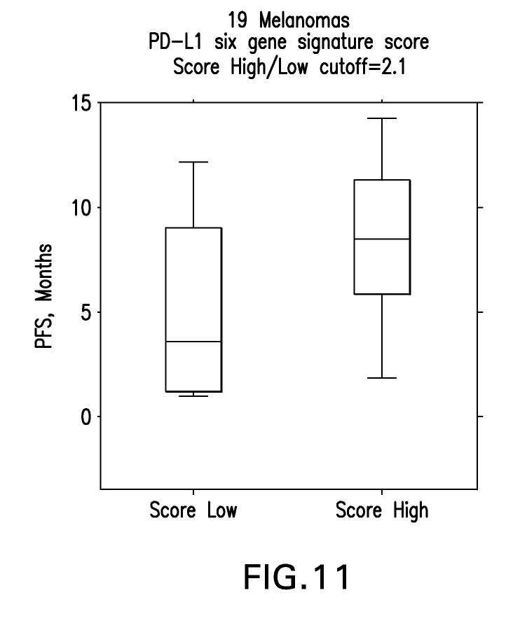

As shown in FIG. 10, when 2.1 was chosen as a reference (cutoff) score, the response rate was greater than 60% in patients from the 19 patient cohort who were classified as biomarker positive (PD-L1 gene signature score at or higher than the cut-off) but less than 30% in patients classified as biomarker negative (PD-L1 gene signature score below the cut-off). Also, the mean length of PFS in this cohort was significantly longer in biomarker patients (i.e., score at or greater than 2.1) than in biomarker negative patients (i.e., score less than 2.1).

Table 7 provides a brief description of the sequences in the sequence listing.

| SEQ ID NO: | Description |
|---|---|
| 1 | hPD-1.08A light chain CDR1 |
| 2 | hPD-1.08A light chain CDR2 |
| 3 | hPD-1-08A light chain CDR3 |
| 4 | hPD-1.08A heavy chain CDR1 |
| 5 | hPD-1.08A heavy chain CDR2 |
| 6 | hPD-1.08A heavy chain CDR3 |
| 7 | hPD-1.09A light chain CDR1 |
| 8 | hPD-1.09A light chain CDR2 |
| 9 | hPD-1.09A light chain CDR3 |
| 10 | hPD-1.09A heavy chain CDR1 |
| 11 | hPD-1.09A heavy chain CDR2 |
| 12 | hPD-1.09A heavy chain CDR3 |
| 13 | 109A-H heavy chain variable region |
| 14 | 409A-H heavy chain full length |
| 15 | K09A-L-11 light chain variable region |
| 16 | K09A-L-16 light chain variable region |
| 17 | K09A-L-17 light chain variable region |
| 18 | K09A-L-11 light chain full length |
| 19 | K09A-L-16 light chain full length |
| 20 | K09A-L-17 light chain full length |
| 21 | MK-3475 Heavy chain |
| 22 | MK-3475 Light chain |
| 23 | Nivolumab Heavy chain |
| 24 | Nivolumab light chain |

### REFERENCES

1. Sharpe, A. H, Wherry, E. J., Ahmed R., and Freeman G. J. The function of programmed cell death 1 and its ligands in regulating autoimmunity and infection. Nature Immunology (2007); 8:239-245.
2. Dong H et al. Tumor-associated B7-H1 promotes T-cell apoptosis: a potential mechanism of immune evasion. Nat Med. 2002 August; 8(8):793-800.
3. Yang et al. PD-1 interaction contributes to the functional suppression of T-cell responses to human uveal melanoma cells in vitro. Invest Ophthalmol Vis Sci. 2008 June; 49(6 (2008): 49: 2518-2525.
4. Ghebeh et al. The B7-H1 (PD-L1) T lymphocyte-inhibitory molecule is expressed in breast cancer patients with infiltrating ductal carcinoma: correlation with important high-risk prognostic factors. Neoplasia (2006) 8: 190-198.
5. Hamanishi J et al. Programmed cell death 1 ligand 1 and tumor-infiltrating CD8+ T lymphocytes are prognostic factors of human ovarian cancer. Proceeding of the National Academy of Sciences (2007): 104: 3360-3365.
6. Thompson R H et al. Significance of B7-H1 overexpression in kidney cancer. Clinical genitourin Cancer (2006): 5: 206-211.
7. Nomi, T. Sho, M., Akahori, T., et al. Clinical significance and therapeutic potential of the programmed death-1 ligand/programmed death-1 pathway in human pancreatic cancer. Clinical Cancer Research (2007); 13:2151-2157.
8. Ohigashi Y et al. Clinical significance of programmed death-1 ligand-1 and programmed death-1 ligand 2 expression in human esophageal cancer. Clin. Cancer Research (2005): 11: 2947-2953.
9. Inman et al. PD-L1 (B7-H1) expression by urothelial carcinoma of the bladder and BCG-induced granulomata: associations with localized stage progression. Cancer (2007): 109: 1499-1505.
10. Shimauchi T et al. Augmented expression of programmed death-1 in both neoplasmatic and nonneoplastic CD4+ T-cells in adult T-cell Leukemia/Lymphoma. Int. J. Cancer (2007): 121:2585-2590.
11. Gao et al. Overexpression of PD-L1 significantly associates with tumor aggressiveness and postoperative recurrence in human hepatocellular carcinoma. Clinical Cancer Research (2009) 15: 971-979.
12. Nakanishi J. Overexpression of B7-H1 (PD-L1) significantly associates with tumor grade and postoperative prognosis in human urothelial cancers. Cancer Immunol Immunother. (2007) 56: 1173-1182.
13. Hino et al. Tumor cell expression of programmed cell death-1 is a prognostic factor for malignant melanoma. Cancer (2010): 00: 1-9.
14. Ghebeh H. Foxp3+ tregs and B7-H1+/PD-1+ T lymphocytes co-infiltrate the tumor tissues of high-risk breast cancer patients: implication for immunotherapy. BMC Cancer. 2008 Feb. 23; 8:57.
15. Ahmadzadeh M et al. Tumor antigen-specific CD8 T cells infiltrating the tumor express high levels of PD-1 and are functionally impaired. Blood (2009) 114: 1537-1544.
16. Thompson R H et al. PD-1 is expressed by tumor infiltrating cells and is associated with poor outcome for patients with renal carcinoma. Clinical Cancer Research (2007) 15: 1757-1761.

All references cited herein are incorporated by reference to the same extent as if each individual publication, database entry (e.g. Genbank sequences or GeneID entries), patent application, or patent, was specifically and individually indicated to be incorporated by reference. This statement of incorporation by reference is intended by Applicants, pursuant to 37 C.F.R. §1.57(b)(1), to relate to each and every individual publication, database entry (e.g. Genbank sequences or GeneID entries), patent application, or patent, each of which is clearly identified in compliance with 37 C.F.R. §1.57(b)(2), even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

1. A method for testing a tumor for the presence or absence of a biomarker that predicts response to treatment with a PD-1 antagonist, which comprises:
obtaining a sample from the tumor, measuring the raw RNA expression level in the tumor sample for each gene in a PD-L1 gene signature;
normalizing each of the measured raw RNA expression levels; and
calculating the arithmetic mean of the normalized RNA expression levels for each of the genes to generate a score for the PD-L1 gene signature;
wherein the PD-L1 gene signature comprises PD-L1, PD-L2, STAT1, LAG3, CXCL10, and CLEC10a.

2. The method of claim 1, wherein the method further comprises:
comparing the calculated score to a reference score for the PD-L1 gene signature; and
classifying the tumor as biomarker positive or biomarker negative;
wherein if the calculated score is equal to or greater than the reference score, then the tumor is classified as biomarker positive, and if the calculated PD-L1 gene signature score is less than the reference PD-L1 gene signature score, then the tumor is classified as biomarker negative.

3. A method for treating a subject having a tumor which comprises:
determining if the tumor is positive or negative for a PD-L1 gene signature biomarker; and
administering to the subject a PD-1 antagonist if the tumor is positive for the biomarker; or
administering to the subject a cancer treatment that does not include a PD-1 antagonist if the tumor is negative for the biomarker;
wherein the PD-L1 gene signature comprises PD-L1, PD-L2, STAT1, LAG3, CXCL10, and CLEC10a.

4. The method of claim 3, wherein the determining step comprises:
obtaining a sample from the subject's tumor;
sending the tumor sample to a laboratory with a request to test the sample for the presence or absence of a PD-L1 gene signature biomarker; and
receiving a report from the laboratory that states whether the tumor sample is biomarker positive or biomarker negative.

5. A method for treating a subject having a tumor which comprises:
obtaining a sample from the tumor;
measuring the raw RNA expression level in the tumor sample for each gene in a PD-L1 gene signature;
normalizing each of the measured raw RNA expression levels;
calculating the arithmetic mean of the normalized RNA expression levels for each of the genes to generate a score for the PD-L1 gene signature; and
administering to the subject a PD-1 antagonist if the calculated score is equal to or greater than a reference score for the PD-L1 gene signature; or
administering to the subject a cancer therapy that does not include a PD-1 antagonist if the calculated score is less than the reference score;
wherein the PD-L1 gene signature comprises PD-L1, PD-L2, STAT1, LAG3, CXCL10, and CLEC10a.

6. The method of claim 2, wherein the gene signature consists of PD-L1, PD-L2, STAT1, LAG3, CXCL10 and CLEC10a.

7. The method of claim 2, wherein the PD-1 antagonist is nivolumab or MK-3475.

8. The method of claim 2, wherein the tumor sample is a melanoma tumor sample and the PD-1 antagonist is MK-3475.

9-12. (canceled)

13. A drug product which comprises a pharmaceutical composition and prescribing information, wherein the pharmaceutical composition comprises a PD-1 antagonist and at least one pharmaceutically acceptable excipient and the prescribing information states that the pharmaceutical composition is indicated for use in a subject who has a tumor that tests positive for a PD-L1 gene signature biomarker, wherein the gene signature consists of PD-L1, PD-L2, STAT1, LAG3, CXCL10, and CLEC10a.

14. A kit for assaying a tumor sample to determine a PD-L1 gene signature score for the tumor sample, wherein the kit comprises a first set of probes for detecting expression of each gene in the PD-L1 gene signature, wherein the PD-L1 gene signature comprises PD-L1, PD-L2, STAT1, LAG3, CXCL10, and CLEC10a.

15. The kit of claim 14, which further comprises a second set of probes for detecting target transcripts expressed in the tumor sample be a set of normalization genes.

16. The kit of claim 14, wherein the first set of probes is designed to detect expression of each of the following transcripts: NM_014143 for PD-L1, NM_025239 for PD-L2, NM_007315 for STAT1, NM_002286 for LAG3, NM_001565 for CXCL10, and NM_182906 for CLEC10a.
